(19) United States
(12) Patent Application Publication
(10) Pub. No.: US 2025/0171840 A1
(43) Pub. Date: May 29, 2025

(54) THREE-PHASE NESTED AMPLIFICATION AND MULTI-PHASIC DETECTION

(71) Applicant: Cepheid, Sunnyvale, CA (US)

(72) Inventors: Soumitesh Chakravorty, Santa Clara, CA (US); Burcu Ozay, Sunnyvale, CA (US)

(73) Assignee: Cepheid, Sunnyvale, CA (US)

(21) Appl. No.: 18/888,017

(22) Filed: Sep. 17, 2024

Related U.S. Application Data
(60) Provisional application No. 63/539,080, filed on Sep. 18, 2023.

(51) Int. Cl. C12Q 1/686; B01L 7/00

(52) U.S. Cl. CPC C12Q 1/686; B01L 7/52; B01L 2200/026; B01L 2200/16; B01L 2300/0681; C12Q 2600/16

(57) ABSTRACT

The present disclosure provides three-phase, nested amplification methods that facilitate multiplex amplification and multi-phasic detection, particularly multiplex amplification assays that employ target-specific melt probes and/or high-resolution melt (HRM) analysis using an intercalating dye.

### CROSS-REFERENCE TO RELATED APPLICATIONS

This application claims the benefit of U.S. provisional application No. 63/539,080, filed Sep. 18, 2023, which is hereby incorporated by reference in its entirety.

### FIELD

The present disclosure relates generally to methods for detection of target nucleic acids in multiplex nucleic acid amplification-based assays that can be carried out in cartridges. The methods are applicable to detection of, e.g., mutations such as those that confer drug resistance as well as to detection of multiple different organisms in a single assay cartridge.

### BACKGROUND

Extensively drug-resistant tuberculosis (XDR-TB) is a form of tuberculosis caused by bacteria that are resistant to some of the most effective anti-TB drugs. XDR-TB strains have arisen after the mismanagement of individuals with multidrug-resistant TB (MDR-TB).

Almost one in four people in the world is infected with TB bacteria. Only when the bacteria become active do people become ill with TB. Bacteria become active as a result of anything that can reduce the person's immunity, such as HIV, advancing age, or some medical conditions. TB can usually be treated with a course of four standard, or first-line, anti-TB drugs (i.e., isoniazid, rifampin, ethambutol, and pyrazinamide). If these drugs are misused or mismanaged, multidrug-resistant TB (MDR-TB) can develop. MDR-TB takes longer to treat with second-line drugs (“SLIDS,” i.e., fluoroquinolones (FLQ) and amikacin, kanamycin, or capreomycin or new generation oral regimens Bedaquline, Pretomanid and Linezolid), which are more expensive and have more side-effects. XDR-TB can develop when these second-line drugs are also misused or mismanaged and become ineffective. The World Health Organization (WHO) has defined XDR-TB as MDR-TB that is resistant to at least one fluoroquinolone and a SLID. Recently, the oral drugs bedaquiline (BDQ) and linezolid (LZD) have replaced SLIDs as the preferred regimen for treating drug-resistant TB and the definition of XDR-TB has changed accordingly (resistant to FLQ and any of the oral drugs).

TB drug resistance genes have been identified and the presence of Rifampin, Isoniazid and FLQ resistance mutations can be detected in sputum. The mechanisms of resistance to BDQ and LZD are not fully understood currently. However, From the evidence generated thus far, ˜90% of observed LZD resistance can be attributed to a single mutation in the rplC gene (C154R), with remaining ˜2-10% of LZD resistance due to mutations in the rrl (23S rRNA) gene. Resistance to BDQ may occur due to multiple mutations spread across several genes; among which mutations in the atpE and Rv0678 genes are the most critical. The atpE gene contains mutations in two hot spot regions at the codons 28 and 63 associated with BDQ-R, with other mutations described in the codons 59, 61 and 66 with possible BDQ-R association. BDQ-R mutations in the Rv0678 gene are however more widespread and can occur anywhere within the 498 bp gene.

### SUMMARY

Various embodiments contemplated herein may include, but need not be limited to, one or more of the following:

Embodiment 1: A method for detecting target nucleic acids in a sample by nucleic acid amplification, the method comprising: contacting sample nucleic acids with a set of preamplification primer pairs for amplifying target nucleic acids, wherein the target nucleic acids comprise a first set of target nucleic acids and a second target nucleic acid or set of target nucleic acids; subjecting the sample nucleic acids and preamplification primer pairs, in solution, to amplification conditions to amplify any target nucleic acids present in the sample nucleic acids to produce a set of double-stranded amplicons; contacting, in solution, at least a first portion of the double-stranded amplicons with a first set of nested primers and a first set of target-specific probes to form a first reaction mixture; subjecting the first reaction mixture to asymmetric or symmetric amplification conditions in a first amplification to amplify any of the first set of target nucleic acids that are present and conducting a first detection comprising melt analysis or real-time analysis respectively, to detect the presence of any of the first set of target nucleic acids that are present; contacting, in solution, at least a second portion of the double-stranded amplicons with: a second set of nested, symmetric primers for amplifying the second target nucleic acid and a DNA intercalating dye; or a second set of nested symmetric or asymmetric primers for amplifying the second set of target nucleic acids and a second set of target-specific real-time or melt probes, respectively; to form a second reaction mixture; subjecting the second reaction mixture to amplification conditions in a second amplification to amplify the second target nucleic acid or the second set of target nucleic acids, if present, and conducting a second detection comprising: high-resolution melt analysis to detect the presence of the second target nucleic acid, if present; or melt analysis or real-time analysis to detect the presence of any of the second set of target nucleic acids that are present; wherein the first amplification and first detection and the second amplification and detection are sequential but can be carried out in any order.

Embodiment 2: A cartridge for detecting target nucleic acids in a sample, the cartridge comprising: a cartridge body comprising a plurality of chambers therein, wherein the plurality of chambers includes: a sample chamber having at least a fluid outlet in fluid communication with another chamber of the plurality; an optional lysis chamber in fluidic communication with the sample chamber, optionally wherein the sample chamber and lysis chamber are the same; a preamplification reagent chamber comprising a first set of preamplification primer pairs for amplifying target nucleic acids, wherein the target nucleic acids comprise a first set of target nucleic acids and a second set of target nucleic acids; a first reagent chamber comprising a first set of nested primers and a first set of target-specific probes for conducting a first amplification; and a second reagent chamber comprising a second set of nested primers, wherein: the second set of nested primers comprises symmetric primers for amplifying a second target nucleic acid, and the second reagent chamber additionally comprises a DNA intercalating dye; or the second set of nested primers comprises primers for amplifying a second set of target nucleic acids and a second set of target-specific probes; a reaction vessel fluidically coupled to the plurality of chambers of the cartridge body and configured for: i) amplification of nucleic acid and ii) detection and identification of one or a plurality of amplification products via melt analysis and/or real-time PCR, wherein the cartridge is configured to carry out multi-phasic detection; and a filter disposed in a fluidic path between the lysis chamber, if present, or the sample chamber, and the reaction vessel.

Embodiment 3: The cartridge of embodiment 2, wherein the lysis chamber comprises one or more lysis reagents for releasing nucleic acid.

Embodiment 4: The cartridge of embodiment 2 or embodiment 3, wherein the sample chamber and the lysis chamber are the same.

Embodiment 5: The cartridge of any one of embodiments 2-4, wherein the cartridge is a Clinical Laboratory Improvement Amendments (CLIA)-compliant cartridge.

Embodiment 6: A cartridge-based method for detecting target nucleic acids in a sample in a cartridge according to embodiment 2, the method comprising: placing the sample in the sample chamber of the cartridge; if the sample comprises cells, lysing cells in the sample with one or more lysis reagents present within at least one of the plurality of chambers or capturing the cells in a filter within the cartridge and lysing the cells by means of ultrasonication to release sample nucleic acids; if the sample comprises cell-free nucleic acids, capturing the free nucleic acids in a nucleic acid capture chamber and eluting the captured nucleic acid after washing to remove impurities; contacting sample nucleic acids with preamplification primer pairs within the reaction vessel; subjecting the sample nucleic acids and preamplification primer pairs, in solution, to amplification conditions to amplify any target nucleic acids present in the sample nucleic acids to produce a set of double-stranded amplicons; causing the double-stranded amplicons to flow into a first one of the plurality of chambers; drawing, from the first one of the plurality of chambers, a first portion of the double-stranded amplicons and contacting, in solution, said first portion with a first set of nested primers and a first set of target-specific probes to form a first reaction mixture within the reaction vessel; subjecting the first reaction mixture to amplification conditions in a first amplification to amplify any of the first set of target nucleic acids that are present and conducting a first detection comprising melt analysis or real-time analysis to detect the presence of any of the first set of target nucleic acids that are present; causing the first reaction mixture to flow into at least one of the plurality of chambers, and washing the reaction vessel; drawing, from the first one of the plurality of chambers, a second portion of the double-stranded amplicons and contacting, in solution, said second portion of the double-stranded amplicons with: a second set of nested, symmetric primers for amplifying the second target nucleic acid and a DNA intercalating dye; or a second set of nested primers for amplifying the second set of target nucleic acids and a second set of target-specific probes; to form a second reaction mixture within the reaction vessel; subjecting the second reaction mixture to amplification conditions in a second amplification to amplify the second target nucleic acid or the second set of target nucleic acids, if present, and conducting a second detection comprising: high-resolution melt analysis to detect the presence of the second target nucleic acid, if present; or melt analysis or real-time analysis to detect the presence of any of the second set of target nucleic acids that are present; wherein the first amplification and first detection and the second amplification and detection are sequential but can be carried out in any order.

Embodiment 7: The method of embodiment 1, cartridge of any one of embodiments 2-5, or the cartridge-based method of embodiment 6, wherein the second set of nested primers comprises symmetric primers for amplifying the second target nucleic acid and is accompanied by the DNA intercalating dye, and said high-resolution melt analysis is conducted, or said cartridge is configured to conduct high-resolution melt analysis, to detect the presence of the second target nucleic acid, if present.

Embodiment 8: The method of embodiment 1, cartridge of any one of embodiments 2-5, or the cartridge-based method of embodiment 6, wherein the second set of nested primers comprises asymmetric primers for amplifying the second set of target nucleic acids and is accompanied by the second set of target-specific probes, which are melt detection probes, and melt analysis is conducted, or said cartridge is configured to conduct melt analysis, to detect the presence of any of the second set of target nucleic acids that are present.

Embodiment 9: The method of embodiment 1, cartridge of any one of embodiments 2-5, or the cartridge-based method of embodiment 6, wherein the second set of nested primers comprises symmetric primers for amplifying the second set of target nucleic acids and is accompanied by the second set of target-specific probes, which are real-time probes, and real-time analysis is conducted, or said cartridge is configured to conduct real-time analysis, to detect the presence of any of the second set of target nucleic acids that are present.

Embodiment 10: The method, cartridge, or cartridge-based method of any one of embodiments 8-9, wherein the method detects between 10 and 20 target nucleic acids in each of the first or second reaction mixture or the cartridge is configured to detect between 10 and 20 target nucleic acids in each of the first or second reaction mixtures.

Embodiment 11: The method, cartridge, or cartridge-based method of embodiment 10, wherein the method detects between 18 and 20 target nucleic acids in each of the first or second reaction mixture or the cartridge is configured to detect between 18 and 20 target nucleic acids in each of the first or second reaction mixture.

Embodiment 12: The method, cartridge, or cartridge-based method of embodiment 11, wherein the method detects between 36 and 40 target nucleic acids in a single cartridge or the cartridge is configured to detect between 36 and 40 target nucleic acids in a single cartridge.

Embodiment 13: The cartridge of any one of embodiments 10-12, wherein the reaction vessel comprises one reaction chamber, and detection of the target nucleic acids is within the one reaction chamber.

Embodiment 14: The cartridge of any one of embodiments 10-12, wherein the reaction vessel comprises up to 4 reaction chambers, and detection of the target nucleic acids is within the up to 4 reaction chambers.

Embodiment 15: The method, cartridge, or cartridge-based method of any one of embodiments 8-10, wherein the preamplification primers comprise primers specific for one or more of the following drug resistance genes: amikacin resistance genes rrs; aminoglycoside resistance genes rrs and eis; bedaquiline resistance genes atpE and Rv0678; fluoroquinolone resistance genes gyrA and gyrB; capreomycin resistance genes gidB, rrs, and tlyA; clofazimine resistance gene Rv0678; delamanid resistance genes fbiA and ddn; ethionamide resistance genes inhA promoter and ethA; ethambutol resistance gene embB; isoniazid resistance genes fabG1, inhA promoter and katG; linezolid resistance genes rplC and rrl; pyrazinamide resistance gene pncA; rifampin resistance gene rpoB; and streptomycin resistance genes gidB, rrs, and rpsL.

Embodiment 16: The method, cartridge, or cartridge-based method of any one of embodiments 8-10, wherein the preamplification primers comprise primers specific for one or more of the rpoB gene (targeting rpoB RRDR comprising codons 426 through 452, and mutations in the codons 170 and 491), IS6110 gene, IS1081 gene, fabG1 gene, inhA promoter, katG gene, gyrA gene, gyrB gene, pncA gene, rplC gene, rrl gene, atpE gene and Rv0678 gene of Mycobacterium tuberculosis.

Embodiment 17: The method, cartridge, or cartridge-based method of embodiment 16, wherein the preamplification primers comprises primers specific for each of the rpoB gene (targeting rpoB RRDR comprising codons 426 through 452, and mutations in the codons 170 and 491), IS6110 gene, IS1081 gene, fabG1 gene, inhA promoter, katG gene, gyrA gene, gyrB gene, pncA gene, rplC gene, rrl gene, atpE gene and Rv0678 gene of Mycobacterium tuberculosis.

Embodiment 18: The method, cartridge, or cartridge-based method of embodiment 16 or embodiment 17, wherein the first set of nested primers and the first set of target-specific probes comprise primers and probes specific for one or more of the rpoB gene (targeting rpoB RRDR comprising codons 426 through 452, and mutations in the codons 170 and 491), IS6110 gene, IS1081 gene, fabG1 gene, inhA promoter, katG gene, gyrA gene, and gyrB gene.

Embodiment 19: The method, cartridge, or cartridge-based method of embodiment 17, wherein the first set of nested primers and the first set of target-specific probes comprise primers and probes specific for each of the rpoB gene (targeting rpoB RRDR comprising codons 426 through 452, and mutations in the codons 170 and 491), IS6110 gene, IS1081 gene, fabG1 gene, inhA promoter, katG gene, gyrA gene, and gyrB gene.

Embodiment 20: The method, cartridge, or cartridge-based method of any one of embodiments 16-19, wherein the second set of nested, symmetric primers is specific for the rpo0678 gene of Mycobacterium tuberculosis.

Embodiment 21: The method, cartridge, or cartridge-based method of any one of embodiments 16-19, wherein the intercalating dye comprises SYBR green.

Embodiment 22: The method, cartridge, or cartridge-based method of embodiment 16 or embodiment 17, wherein the first set of nested primers and the first set of target-specific probes comprise primers and probes specific for one or more of the rpoB RRDR, and rpoB codon 170 targets of the rpoB gene, IS6110 gene, IS1081 gene, inhA promoter, and katG gene of Mycobacterium tuberculosis.

Embodiment 23: The method, cartridge, or cartridge-based method of embodiment 22, wherein the first set of nested primers and the first set of target-specific probes comprise primers and probes specific for each of the rpoB RRDR, and rpoB codon 170 targets of the rpoB gene, IS6110 gene, IS1081 gene, inhA promoter, and katG gene of Mycobacterium tuberculosis.

Embodiment 24: The method, cartridge, or cartridge-based method of any one of embodiments 16-19, 22, and 23, wherein the second set of nested primers and the second set of target-specific probes comprise primers and probes specific for one or more of the rpoB codon 491 target of the rpoB gene, fabG1 gene, gyrA gene, and gyrB gene of Mycobacterium tuberculosis.

Embodiment 25: The method, cartridge, or cartridge-based method of embodiment 24, wherein the second set of nested primers and the second set of target-specific probes comprise primers and probes specific for each of the rpoB codon 491 target of the rpoB gene, fabG1 gene, gyrA gene, and gyrB gene of Mycobacterium tuberculosis.

Embodiment 26: The method, cartridge, or cartridge-based method of embodiments 8-25, wherein at least one of the primers and or probe(s) comprises a detectable label.

Embodiment 27: The method, cartridge, or cartridge-based method of embodiment 26, wherein at least one probe, optionally each probe, comprises a fluorescent dye and a quencher molecule.

Embodiment 28: The method, cartridge, or cartridge-based method of embodiments 8-27, wherein the method employs, and/or the cartridge comprises, a primer pair that selectively hybridizes to an exogenous control and/or an endogenous control, wherein the exogenous control is a sample processing control, and wherein the endogenous control is a sample adequacy control.

Embodiment 29: The method, cartridge, or cartridge-based method of embodiments 8-28, wherein the amplification comprises isothermal amplification.

Embodiment 30: The method, cartridge, or cartridge-based method of embodiments 8-28, wherein the amplification comprises non-isothermal amplification, optionally by thermal cycling or temperature oscillation.

Embodiment 31: The method of embodiment 1 or the cartridge-based method of embodiment 6, wherein the sample is an unprocessed, or digested, decontaminated and concentrated sputum sample, a nasal aspirate sample, a nasal wash sample, a nasal swab sample, a nasopharyngeal swab sample, a saliva sample, an oropharyngeal swab sample, a throat swab sample, a bronchoalveolar lavage sample, a bronchial aspirate sample, a bronchial wash sample, an endotracheal aspirate sample, an endotracheal wash sample, a tracheal aspirate sample, a nasal secretion sample, a mucus sample, a pleural effusion sample, a cerebrospinal fluid sample, a stool sample, a tissue biopsy sample, a breath sample, or a combination thereof.

Embodiment 32: The method of embodiment 1 or the cartridge-based method of embodiment 6, wherein said detecting is performed at the same facility where the sample was collected from a subject.

Embodiment 33: The method or cartridge-based method of embodiment 32, wherein the method is a point-of-care method.

Embodiment 34: The method of embodiment 1 or the cartridge-based method of embodiment 6, wherein the method is carried out in a hospital, an urgent care center, an emergency room, a physician's office, a health clinic, or a home.

Embodiment 35: The method of embodiment 1 or the cartridge-based method of embodiment 6, wherein the method is a Clinical Laboratory Improvement Amendments (CLIA)-waived test.

Embodiment 36: The cartridge-based method of embodiment 6, wherein the cartridge is a Clinical Laboratory Improvement Amendments (CLIA)-compliant cartridge, is operated in compliance with CLIA, is operated by a CLIA-compliant laboratory, or is operated in a CLIA-compliant location.

Embodiment 37: The method of embodiment 1, the cartridge of embodiment 2, or the cartridge-based method of embodiment 6, wherein the method comprises, and/or the cartridge facilitates, detection of a target nucleic acid in the sample within 150 minutes, within 140 minutes, within 130 minutes, or within 120 minutes of collecting the sample from the subject.

Embodiment 38: The cartridge of embodiment 2 or the cartridge-based method of embodiment 6, wherein the cartridge facilitates, and/or the method comprises, detection of a target nucleic acid in the sample within 130 minutes, within 120 minutes, or within 110 minutes from the time the sample is placed in a cartridge.

Embodiment 39: The cartridge of embodiment 3 or method of embodiment 6, wherein the one or more lysis reagents comprise a chaotropic agent, a chelating agent, a buffer, and a detergent.

Embodiment 40: The cartridge or method of embodiment 39, wherein the chaotropic agent is selected from guanidinium thiocyanate, guanidinium hydrochloride, alkali perchlorate, alkali iodide, urea, formamide, or a combination thereof.

Embodiment 41: The cartridge or method of embodiment 39 or 40, wherein the one or more lysis reagents comprise a guanidinium compound, sodium hydroxide, EDTA, a buffer, and a detergent.

Embodiment 42: The cartridge of embodiment 2, wherein the filter is configured to bind the nucleic acid to be analyzed.

Embodiment 43: The cartridge of embodiment 42, wherein the filter comprises glass fibers and optionally a polymeric binder, or the glass fibers are optionally modified with a DNA binding ligand, optionally an alkylamine, a cycloalkylamine, an alkyloxy amine, a polyamine moiety, an arylamine, an intercalating agent, a DNA groove binder, a peptide, an amino acid, a protein, or a combination thereof.

Embodiment 44: The cartridge of embodiment 42 or embodiment 43, wherein the filter comprises a 500 micron to 2000 microns thick glass fiber disk having a pore size of 0.2 microns to 1 micron.

Embodiment 45: The cartridge of any one of embodiments 42-44, wherein the filter is configured to bind unwanted material and allow the nucleic acid to pass through.

Embodiment 46: The cartridge of embodiment 3 or method of embodiment 6, wherein the cartridge further comprises a binding reagent, wash reagent, eluting reagent, or a combination thereof.

Embodiment 47: The cartridge or method of embodiment 46, wherein the eluting reagent comprises ammonia or an alkali metal hydroxide.

Embodiment 48: The cartridge or method of embodiment 46 or embodiment 47, wherein the eluting reagent has a pH above about 9, above about 10, or above about 11.

Embodiment 49: The cartridge or method of any one of embodiments 46-48, wherein the eluting reagent comprises a polyanion, optionally a carrageenan, a carrier nucleic acid, or i-carrageenan and KOH.

Embodiment 50: The cartridge of embodiment 2 or method of embodiment 6, wherein the reaction vessel comprises up to 4 reaction chambers.

Embodiment 51: The cartridge of embodiment 2 or method of embodiment 6, wherein the reaction vessel comprises one reaction chamber.

Embodiment 52: The cartridge of embodiment 2 or method of embodiment 6, wherein at least one of the plurality of chambers comprises one or more lyophilized reagents.

Embodiment 53: The cartridge or method of embodiment 52, wherein the one or more lyophilized reagents is/are in the form of one or more beads.

Embodiment 54: The cartridge or method of embodiment 52 or embodiment 53, wherein the one or more lyophilized reagents are selected from primers, probes, a salt, dNTPs, a thermostable polymerase, a reverse transcriptase, or a combination thereof.

Embodiment 55: The cartridge or method of embodiment 54, wherein the one or more lyophilized reagents comprise lyophilized primers and probes.

Embodiment 56: The cartridge of embodiment 2 or method of embodiment 6, wherein reagents and components in the reaction vessel are in solution.

### BRIEF DESCRIPTION OF THE DRAWINGS

FIGS. 1A-1C show an overview of a sample cartridge with a valve assembly configured for performing different sample processing steps, including chemical lysing of targets, which is configured for PCR and optional integrated nucleic acid analysis. FIG. 1A shows the sample cartridge body with reaction vessel, FIG. 1B shows an exploded view of the sample cartridge, and FIG. 1C shows components of the valve assembly, in accordance with some embodiments.

FIGS. 2A-2C show illustrative, but non-limiting, embodiments of the modules and systems (e.g., processing units) for the PCR detection and/or quantification and optional integrated nucleic acid analysis for the biomarker panel. FIG. 2A illustrates a module configured to receive and interact with the valve assembly of the cartridge to operate the cartridge to facilitate sample preparation and analysis. FIG. 2B illustrates a processing unit (e.g., analytical testing unit) of the module that interacts with the fluid sample in the reaction vessel to facilitate sample processing and analytical testing (e.g., PCR and, optionally, nucleic acid analysis) for the biomarker panel. FIG. 2C illustrates an analytical system having multiple such modules within an enclosure so as to receive multiple sample cartridges therein for testing of the biomarker panel and/or various other targets or panels.

FIG. 3 illustrates various valve assemblies A, B, C, D, each suited for one or more types of target lysing, any of which may be used in a respective sample cartridge.

FIG. 4 shows the GENEXPERT® cartridge layout for the study of Example 1.

FIG. 5 shows the experimental set-up for the study of Example 1, showing the biomarkers for drug resistance and the phases in which they were detected.

FIGS. 6A-6D show real-time PCR signals (15 signals) from the study of Example 1. FIGS. 6A-6C show that all 15 signals were detected in assays of 10,000, 1,000, and 100 cfu/mL Mycobacterium bovis BCG (BCG), respectively. FIG. 6D shows detection of a sample processing control (SPC) real-time signal only from negative controls with no BCG.

FIGS. 7A-7D show melt signals (13 signals; first derivative) from the study of Example 1. FIGS. 7A-7C show that all 13 signals were detected in assays of 10,000, 1,000, and 100 cfu/mL BCG, respectively. FIG. 7D shows that no melt curves were detected from negative controls with no BCG.

FIGS. 8A-8B show real-time signals from two fluorescent probes and EvaGreen from the study of Example 2 for Amplifications 1 and 2 respectively (the second and third phases of the method). FIG. 8A shows the real-time signals that were detected in assays of 10,000 cfu/mL BCG. FIG. 8B shows the results for the negative controls, where only EvaGreen signal was detected, indicating the formation of primer dimers in the absence of any amplified product.

FIGS. 9A-9B show melt signals (first derivative) from the study of Example 2 for Amplification 1 (the second phase of the method). FIG. 9A shows that signals were detected in assays of 10,000 cfu/mL BCG. FIG. 9B shows that no melt curves were detected from negative controls with no BCG.

FIGS. 10A-10B show the results of high-resolution melt (HRM) from the study of Example 2 for Amplification 2 (the third phase of the method). FIG. 10A shows that appropriate melt signals were detected in assays of 10,000 cfu/mL BCG (N1-N3 are three replicates). FIG. 10B shows that appropriate signals were not detected from negative controls with no BCG (N1-N3 are three replicates). The minor melt peaks produced in the negative controls (FIG. 10B) are indicative of primer dimers.

FIGS. 11A-11D show the results from the study of Example 3, in which an 8-plex/10-color assay was performed twice over, in the same cartridge, to generate 18 independent melt and 2 real-time signals in a 10-color instrument, enabling detection of a total of 20 independent signals in a 10-color GENEXPERT® instrument. FIG. 11A shows the signals from the nested PCR-1 melt probes; FIG. 11B shows the signal from the nested PCR-1 real-time probe; FIG. 11C shows the signals from the nested PCR-2 melt probes; and FIG. 11D shows the signal from the nested PCR-2 real-time probe.

### DETAILED DESCRIPTION

The present disclosure describes methods, compositions, devices, and systems that rely on three-phase, nested amplification schemes that facilitate multiplex target nucleic acid amplification (e.g., polymerase chain reaction [PCR]) and multi-phasic detection in a selective and specific manner that is readily automated and can be employed in point-of-care devices. These methods, compositions, devices, and systems enable inter alia the use of target-specific melt probe detection and high-resolution melt (HRM) analysis in a single, automated assay that can be performed in a single assay cartridge.

It is not generally feasible to combine target-specific melt and HRM using intercalating dye in a multiplexed PCR system in a single tube, especially when simultaneous amplification of multiple targets is carried out. Intercalating dyes are non-specific and can bind to any amplicon and hence has not previously been used to determine variants or mutations in genes in a single cartridge. In a multiplexed PCR system, which amplifies several targets at the same time, HRM with an intercalating dye will lead to a very complex and uninterpretable melt profile due to non-specific intercalation of the dye in all the amplicons being amplified in the tube. However, HRM is advantageous to identify unknown mutations in longer amplicons as well as mutations spread over a gene >200 bp, since hybridization oligonucleotide melt probes cannot be easily used to cover such large gene stretches to detect mutations. This disclosure overcomes the challenge to combine both hybridization target-specific melt detection and intercalating dye HRM detection in the same multiplexed PCR system.

Furthermore, attempts at creating multiplexed real-time PCR methods have been plagued by practical issues of simultaneously detecting different nucleic acid sequences in a single sample. A possible approach is to associate different reporter molecules (e.g., fluorescent dyes) to individual amplicons during the PCR reaction which may enable parallel detection of individual reporters by different “colors.” While such approach, in theory, may offer parallelism, it is limited by: (i) the number of different reporter molecules available; (ii) crosstalk from optical signal present in a channel interfering with the optical signal in an adjacent channel and vice versa; and (iii) the availability of imagers and detectors capable of differentiating different signals.

Another possible approach to assaying for a large number of target nucleic acids in a single assay is to divide the biological sample of interest and physically place it, using fluidic systems, into separate, single and isolated amplification chambers. While this approach allows for simultaneous detection of many targets by performing multiple single-plex (i.e., one amplicon per chamber) PCR reactions, it may be suboptimal, since it may reduce the number of target nucleic acid sequences in each chamber which may create stochastic anomalies (Poisson noise) in the acquired data when the original sample has a small concentration. Further, this approach requires complex fluidic handling procedures.

Some benefits of highly multiplexed detection of target nucleic acids in a sample may be achieved through adopting analytical platforms such as DNA microarrays or next-generation DNA sequencers. Microarrays, in particular, are massively-parallel, affinity-based biosensors where target nucleic acids are captured selectively from the same sample at different addressable coordinates (e.g., pixels) on a solid surface. Each addressable coordinate can have a unique capturing DNA or RNA probe, complementary to a target nucleic acid sequence to be detected in the sample. While microarrays can facilitate detection of a large number of targets in a sample, they are semiquantitative and are inferior in terms of limit-of-detection (LOD) and detection dynamic range (DDR), due to their end-point detection nature (i.e., no real-time detection) and the fact that they lack any target amplification.

For many applications, it is advantageous to automate assays for target nucleic acids, which can magnify the challenges discussed above. Assays that can be performed in a single cartridge that can function both as a point-of-care device and/or in a central laboratory are of particular interest. However, assays in an, e.g., 10-color instrument for conducting cartridge-based assays can only detect 10 independent optical signals at one time, which has provided a practical limit on multiplex detection.

The technology described herein overcomes these challenges and finds particular application in situations where it is advantageous to combine the detection of specific, known polymorphisms in same or different genes, with the detection of multiple polymorphisms distributed throughout a single gene. This detection challenge must be met, for example, to identify resistance to the tuberculosis (TB) drugs fluoroquinolone (FLQ), bedaquiline (BDQ), and linezolid (LZD) in a single, automated assay. In an illustrative embodiment, the technology described herein enables the detection of more than 10 targets in a single, cartridge-based assay, with a time-to-result under 2 hours (e.g., about 110 minutes).

### Definitions

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (2002) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

As used herein, the term “gene” encompasses coding sequences, introns, and any associated control sequences that participate in the expression of the coding sequences.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Selective hybridization” or “selective annealing” refers to the binding of a nucleic acid to a target nucleic acid in the absence of substantial binding to other nucleic acids present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, Vol. 152: Guide to Molecular Cloning Techniques, San Diego: Academic Press, Inc. and Sambrook et al. (1989) Molecular Cloning: A Laboratory Manual, 2^{nd}., Vols. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41 (% G+C), when a nucleic acid is in aqueous solution at 1 M NaCl (see, e.g., Anderson and Young, Quantitative Filter Hybridization in Nucleic Acid Hybridization (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonucleotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to “anneal to” or “hybridize to” another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of 5′ end of the DNA sequence to be amplified and a 3′ “downstream primer” or “reverse primer” that hybridizes with 3′ end of the sequence to be amplified. As will be recognized by those of skill in the art, the terms “upstream” and “downstream” or “forward” and “reverse” are not intended to be limiting, but rather provide illustrative orientations in some embodiments.

As used herein, the term “preamplification primer pair” refers to a pair of primers that is employed in an initial amplification, which is followed by at least one further amplification designed to amplify at least one of the same target nucleic acids as in the initial amplification.

As used herein, a “nested primer” selectively hybridizes within an amplicon produced from a previous amplification, such as an initial or “preamplification.”

As used herein, “symmetric primers” produce double-stranded amplicons.

As used herein, “asymmetric primers” produce a nucleic acid strand complementary to that of the nucleic acid strand to which they anneal, i.e., asymmetric primers produce single-stranded amplicons.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable moiety to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

The term “target” is used herein with reference to “target nucleic acids,” as well as “target organisms.” The former refers to nucleic acids to be detected, and the latter refers to organisms to be detected. The term, “target nucleic acid” is generally used herein to refer to a segment of nucleic acid that is defined by a primer pair and that gives rise to an amplicon produced in an amplification reaction; the term “amplification target” is also used herein to refer to this type of target nucleic acid. Primers and probes are also said to “target” nucleic acid sequences, and so these sequences can also be understood as “target nucleic acids.” Additionally, primers and probes are said to “target” or “be specific for” genes. In this usage, the primers and probes can be used to detect the presence of a particular gene by specifically hybridizing to a portion of the gene that indicates its presence. The meaning of “target” and “target nucleic acids” will be clear to one of skill in the art from the context in which the term is employed. In some embodiments, multiple target nucleic acids can be detected to detect a single target organism. In some embodiments, a single target nucleic acid can be detected to detect a single target organism. In some embodiments, an assay can employ multiple target nucleic acids for one or more target organisms and single target nucleic acids for one or more different target organisms.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), helicase-dependent amplification (I), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4 (1): 41-7, U.S. Pat. Nos. 6,027,998; 6,605,451, Barany et al., PCT Publication No. WO 97/31256; Wenz et al., PCT Publication No. WO 01/92579; Day et al., Genomics, 29 (1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web a21romegaega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13 (2): 294-307, and Landegren et al., Science 241:1077-80 (1988), Demidov, V., Expert Rev Mol Diagn. 2002 November; 2 (6): 542-8., Cook et al., J Microbiol Methods. 2003 May; 53 (2): 165-74, Schweitzer et al., Curr Opin Biotechnol. 2001 February; 12 (1): 21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

As used herein, the term “amplification conditions” refers to conditions that promote amplification of a target nucleic acid in the presence of suitable primers.

As used herein, “in solution” means not immobilized on a substrate of any kind, for example, a bead or a surface in a cassette, such as a chamber wall.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

The term “melt curve analysis” refers to the use of the dissociation characteristics of a segment of double-stranded nucleic during heating. Originally, strand dissociation was observed using UV absorbance measurements, but techniques based on fluorescence measurements are now the most common approach. The temperature-dependent dissociation between two DNA-strands can be measured in a “melt assay,” for example, using a DNA-intercalating fluorophore, such as SYBR green or EvaGreen, or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher) can be used to determine the complementarity of the probe to the target nucleic acid sequence.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation and produces a detectable signal (e.g., a fluorescent signal).

The term “quencher,” as used herein generally refers to any organic or inorganic molecule that reduces the level of a detectable signal.

As used herein, the term “detecting” refers to “determining the presence of” an item, such as a nucleic acid sequence.

As used herein, the term “multi-phasic detection” refers to at least two detection steps that are performed sequentially. Each detection step can be carried out in a single reaction chamber or a plurality of reaction chambers.

As used herein, the term “treatment regimen” refers to any medical intervention intended to mitigate the symptoms and/or the pathology of a disorder. The treatment regimen can include one or more actions (e.g., bed rest, increasing fluid intake), non-prescription or prescription medications, supplements, foods, drinks, or the use of medical devices (e.g., a respirator).

As used herein, “Clinical Laboratory Improvement Amendments (CLIA)” refers to The Clinical Laboratory Improvement Amendments of 1988 (CLIA) regulations in effect as of the original filing date of the present application. The CLIA regulations include federal standards applicable to all U.S. facilities or sites that test human specimens for health assessment or to diagnose, prevent, or treat disease. A “CLIA-compliant” test is one that complies with these regulations. “CLIA-waived” tests include tests that does not comply with all of these regulations. For example, CLIA-waived tests include test systems cleared by the U.S. Food and Drug Administration for home use and those tests approved for waiver under the CLIA criteria.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA for a human sample. Nonlimiting exemplary endogenous controls include ABL mRNA, GUSB mRNA, GAPDH mRNA, TUBB mRNA, and UPKla mRNA. In some embodiments, an endogenous control, such as an SAC, is selected that can be detected in the same manner as the target nucleic acid (e.g., RNA) is detected and, in some embodiments, simultaneously with the target nucleic acid (e.g., RNA).

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target nucleic acid (e.g., RNA). In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target nucleic acid (e.g., RNA) is detected and, in some embodiments, simultaneously with the target nucleic acid (e.g., RNA). In some embodiments, the exogenous control is an RNA. In some such embodiments, the exogenous control is an Armored RNA®, which comprises RNA packaged in a bacteriophage protective coat. See, e.g., WalkerPeach et al, Clin. Chem. 45:12: 2079-2085 (1999).

### Three-Phase, Nested Amplification and Multi-Phasic Detection

The present disclosure provides three-phase, nested amplification methods that facilitate multiplex amplification and multi-phasic detection, particularly multiplex amplification assays that employ target-specific melt probes and/or high-resolution melt (HRM) analysis using an intercalating dye. Two illustrative methods that are particularly useful in automated, single-cartridge assays, such as those performed by Cepheid's GeneExpert® system, are described below. These methods share the following common steps.

Sample nucleic acids are first preamplified with preamplification primers that specifically amplify target nucleic acids of interest. The target nucleic acids include two “sets” (i.e., “first” and “second” sets) of target nucleic acids that are detected separately from one another using multi-phasic detection steps can are carried out sequentially (in any order). A “set” of target nucleic acid can include a plurality of target nucleic acids, and one of the sets typically does. In some embodiments, a “set” can consist of just one target nucleic acid. The preamplification is carried out, typically with all reaction components in solution, to produce a set of double-stranded amplicons.

A portion of the double-stranded amplicons (termed the “first portion” for ease of discussion) is combined with a first set of nested primers and a first set of target-specific probes to form a first reaction mixture. The nested primers anneal to sequences present in the double-stranded amplicons that correspond to the first set of target nucleic acids, and amplification, typically in solution, produces copies of one or both strands of the first portion of double-stranded amplicons (asymmetric primers preferentially yield one strand, whereas symmetric primers produce copies of both strands, as in standard polymerase chain reaction (PCR)). These copies can readily be detected by melt probe analysis or real-time PCR analysis using the probes present in the first target detection reaction mixture. This amplification enables convenient multiplex detection of target nucleic acids using melt analysis and/or real-time analysis, which constitutes a first detection step in the method, which employs multi-phasic detection. The term “first” is used in this context is simply to distinguish this step from the “second” detection step described below. These terms are intended to imply that these detections are carried out separately and sequentially as part of multi-phasic detection. These terms are not intended to imply any particular order: the first detection can be carried out first or second in the method, and the second detection can be carried out second or first. Although this method step is discussed in terms of detecting a plurality of target nucleic acids, one of skill in the art appreciates that this method step can also be carried out to detect a single target nucleic acid.

A second portion of the double-stranded amplicons produced upon preamplification is amplified in a second target detection reaction, followed by detection of one or more of the second set of target nucleic acids. The use of the terms “first” and “second” are not meant to imply a particular order of these steps: the amplification of the first reaction mixture can be carried out before, after, or simultaneously with, the amplification of the second reaction mixture (likewise, there is no criticality to the order of formation of the first and second reaction mixtures). The details of the amplification of the second reaction mixtures differ between the two illustrative embodiments of the method described herein.

### Illustrative Method A: Preamplification→Amplification 1→Detection 1→Uniplex Amplification 2→Detection 2 by HRM

In one embodiment, the second reaction mixture includes a second set of nested, symmetric primers for amplifying a single target nucleic acid that makes up the second target nucleic acid “set,” as well as a DNA intercalating dye. High-resolution melt (HRM) analysis is carried out to detect, and optionally characterize, the second target nucleic acid. This detection represents one possible second detection step in an illustrative embodiment of the method.

In some embodiments, the Amplification 1 is carried out with asymmetric primers to produce single-stranded amplicons, and the target-specific probes are melt probes. In this case, Detection 1 can be carried out by melt analysis. The use of asymmetric primers for Amplification 1 provides the advantage of minimal interference from asymmetric single-stranded amplicon carryover from Amplification 1 when all amplification and detection steps in the method are carried out in a single reaction chamber (minor carryover single-stranded amplicons will bind less readily to a DNA intercalating dye than will the double-stranded amplicon produced using symmetric primers in Amplification 2). Reduced carryover gives a cleaner HRM result in Detection 2, as well as higher sensitivity, especially when the second target nucleic acid is large, e.g., like the >500-basepair Rv0678 gene.

### Illustrative Method B: Preamplification→Amplification 1→Detection 1→Amplification 2→Detection 2

In another embodiment, the second reaction mixture includes a second set of nested primers for amplifying the second set of target nucleic acids and a second set of target-specific probes. Melt or real-time analysis is carried out to detect the presence of any of the second set of target nucleic acids that are present. This detection represents another possible second detection step in another illustrative embodiment of the method.

In some embodiments, this method can be carried out to detect a large number of target nucleic acids in a single assay. For example, the preamplification can include 20 different preamplification primer pairs that can generate 20 separate amplicons following a parallel amplification scheme of multiple genes in a single organism or can generate one or more amplicons from a list of 20 target genes present in 20 different organisms; alternatively, a combination of these approaches can be employed to generate one or more amplicons in 2-19 different organisms. A first portion of the amplicons so generated could be further amplified using 10 nested primer pairs to detect 10 target nucleic acids in the Amplification 1/Detection 1, and a second portion of the amplicons generated by the preamplification could be further amplified using another 10 nested primer pairs to detect 10 additional target nucleic acids in Amplification 2/Detection 2. Both the detection phases are performed in a single reaction chamber, the second detection step is performed after removing and washing off the first set of 10 amplicons from the reaction chamber.

Higher levels of multiplexing are possible using combinations of melt and real-time probes. As an example, the table below shows the number of signals that can be detected using method B in an automated, single-cartridge assay, such as that performed by Cepheid's GeneExpert® system, using Taqman probes only, melt probes only, or a combination of both.

TABLE 1
Multiplexing with Melt and Real-Time Probes in Cartridge-
Based Nucleic Acid Amplification with Ten Optical Channels
in a GENEXPERT ® System

| | Total number of signals detectable [symbol] | | |
|---|---|---|---|
| | Theoretical | Theoretical | |
| | Amplification/ | Amplification/ | Theoretical |
| Probe type | Detection 1 | Detection 2 | Total signals |
| Taqman probes only | 10 | 10 | 20 |
| Melt probes only | 30* | 30* | 60* |
| Taqman + Melt | 40* | 40* | 80* |
| probes | | | |

*Assumes that at least 3 independent melt signals can be detected per optical channel, which has been shown in some existing assays [symbol]
The total number of signals detectable changes based on the number of optical channels available. For example, the total number of signals detectable decreases by a half in a system with five optical channels or increases by two times in a system with twenty optical channels.

This degree of multiplexing, especially in an automated system, represents a significant advance in view of various issues with currently available multiplexed PCR methods. For instance, while multiplexing a large number of target amplification reactions (e.g., multiplexed PCR) may be possible, it is not straightforward to detect multiple amplicons simultaneously. Thus far, multiplexed real-time methods, defined as the processes by which one amplifies and detects a plurality of nucleic acid sequences simultaneously in a single reaction chamber, have been implemented for a small number of amplicons. It is of great interest to efficiently multiplex the assays in the same reaction volume and allow for multiple concurrent target amplification and detection in the same reaction chamber. Such an approach may not only better utilize the original DNA sample, but also significantly reduce any complexities associated with the fluidics and liquid-handling procedures for running multiple single-plex reactions.

In some embodiments, the methods described herein can include multiplex amplification reactions, which can be designed to detect 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more target nucleic acids per amplification reaction mixture. This degree of multiplexing can be achieved by using primers and probes that do not substantially cross-react or bind off-target and, in some, embodiments, by combining melt and real-time detection of the same amplification reaction. The methods described herein further extend the number of targets that can be detected in a single assay by using a preamplification reaction, with two subsequent separate amplification reactions, and multi-phasic (bi-phasic in this case) detection. The two illustrative embodiments described above can reduce signal interference and artifacts due to carryover and is readily automated in, e.g., a cartridge-based format.

In some embodiments, to increase the number of target nucleic acids detected per channel, the following approaches can be used: (i) Taqman and melt probes can be combined in the same channel using melt probe with a Tₘ below annealing temperature (no amplification curve); and (ii) several melt target nucleic acids in one channel; the melt window for each target will be dependent on the sequence variation of the target.

Because a large number of oligonucleotides combined in one reaction mixture can lead to unwanted interactions between them, in some embodiments, modified nucleotides can be used to reduce primer-primer interactions.

### Multiplex Detection of Resistance to Tuberculosis Drugs

In one embodiment, Method A, described above, can be employed to detect resistance to the anti-TB drugs FLQ, LZD and BDQ by targeting, e.g., 6-7 different genes which can be co-amplified in a single cartridge. The presence or absence of mutations in these genes can be detected by monitoring Tₘ values from melt probes for all but one of selected target genes and performing HRM using a DNA intercalating dye to assess mutations in a further target gene. This probe melt and SYBR green HRM multiplexed PCR assay can be performed in a single cartridge using three-phase, nested PCR, with multiphasic detection characterized by the following workflow: Preamplification→PCR1→Melt→Uniplex PCR2→HRM. In an illustrative embodiment, the preamplification can include outer primers for all the target genes, gyrA, gyrB, rplC, rrl, atpE, Rv0678 and IS1081 genes (optional; for MTB detection) to preamplify all targets. The PCR1 amplification can include nested, asymmetric primers and target-specific melt probes/detection probes for all the targets except Rv0678. This amplification generates single-stranded DNA (ssDNA) amplicons for all the targets except Rv0678. These single-stranded amplicons can be detected in a first phase of detection by melt analysis of the melt probes. The PCR2 amplification, which can be a simplex amplification for the Rv0678 gene, can include symmetric, nested primers that generates a double-stranded DNA (dsDNA) amplicon for Rv0678. Mutations in this double-stranded DNA amplicon can be detected by HRM in a second phase of detection, which enables the detection of mutations, regardless of where they occur in this gene. The use of asymmetric primers in PCR1, followed by symmetric primers in PCR facilitates HRM analysis in at least two ways: i) it reduces interference from asymmetric ssDNA amplicon carryover from PCR1 (minor carryover ssDNA amplicon will bind less readily to SYBR green) resulting in a cleaner Rv0678 assay-specific HRM result due to preferential double-stranded amplicon generation in a uniplex format in PCR2; and ii) better sensitivity for >500 bp Rv0678 amplicon.

Illustrative primers that can be used in multiplex detection of resistance to tuberculosis drugs. Forward and reverse primers are designated “FOR” and “REV,” respectively.

TABLE 2
Illustrative Primers for Multiplex Detection of Resistance to
Tuberculosis Drugs

| Name | Type | Sequence | Literature reference |
|---|---|---|---|
| IS6110, OUTER-FOR | Primer | AGC GCC GCT TCG GAC CAC CAG | US20220064715A1 |
| | | | PMID: 28851844 |
| IS6110, OUTER-REV | Primer | AGG CGT CGG TGA CAA AGG CCA CGTA | US20220064715A1 |
| | | | PMID: 28851844 |
| IS1081, FOR | Primer | CAG CCC GAC GCC GAA TCA GTT GTT | US20220064715A1 |
| | | | PMID: 28851844 |
| IS1081, OUTER-REV | Primer | GGT GCG GGC GGT GTC GAG GTG | US20220064715A1 |
| | | | PMID: 28851844 |
| RPO-B, FOR1 | Primer | CGT GGA GGC GAT CAC ACC GCA GAC | PMID: 28851844 |
| | | | PMID: 22535987 |
| RPO-B, REV | Primer | AGC TCC AGC CCG GCA CGC TCA CGT | US20220064715A1 |
| | | | PMID: 2885184 4; |
| | | | PMID: 22535987 |
| katG out For | Primer | CCGCTGGAGCAGATGGGCTTG G | US20220064715A1 |
| | | | PMID: 27807153 |
| katG out Rev | Primer | CCA TTT CGT CGG GGT GTT CGT CC | US20220064715A1 |
| | | | PMID: 27807153 |
| INHA out For | Primer | CACGTTACGCTCGTGGACATACCG | US20220064715A1 |
| | | | PMID: 27807153 |
| INHA out Rev | Primer | CGGTAACCAGGACTGAACGGGATACG | US20220064715A |
| | | | PMID: 217807153 |
| IS6110, INNER-FOR | Primer | CGC CGC TTC GGA CCA CCA GCA C | US20220064715A1 |
| | | | PMID: 28851844 |
| IS6110, INNER-REV | Primer | GTG ACA AAG GCC ACG TAG GCG AAC C | US20220064715A1 |
| | | | PMID: 28851844 |
| IS1081, FOR | Primer | CAG CCC GAC GCC GAA TCA GTT GTT | US20220064715A1 |
| | | | PMID: 28851844 |
| IS1081, INNER-REV | Primer | GCC ACC GCG GGG AGT TTG TCG | US20220064715A1 |
| | | | PMID: 28851844 |
| GYRA in For | Primer | GTC GGT TGC CGA GAC CAT GGG C | US20220064715A1 |
| | | | PMID: 21191047 |
| | | | PMID: 27807153 |
| GYRA in Rev | Primer | AGC GAC CAG GGC TGG GCC ATG | US20220064715A1 |
| | | | PMID: 21191047 |
| | | | PMID: 27807153 |
| RPO-B, FOR2 | Primer | ATC AAC ATC CGG CCG GTG GTC GCC | US20220064715A1 |
| | | | PMID: 28851844 |
| | | | PMID: 22535987 |
| RPO-R2A, INNER-REV | Primer | TCA CGT GAC AGA CCG CCG GGC | US20220064715A1 |
| | | | PMID: 28851844 |
| | | | PMID: 22535987 |
| INHA Inn For | Primer | TTA CGC TCG TGG ACA TAC CGA TTT | US20220064715A1 |
| | | CGG | PMID: 27807153 |
| INHA Inn Rev | Primer | GTT TGG CCC CTT CAG TGG CTG TGG | US20220064715A1 |
| | | | PMID: 27807153 |
| KATG in For | Primer | GGC TGG AAG AGC TCG TAT GGC ACC G | US20220064715A1 |
| | | | PMID: 27807153 |
| KATG in Rev | Primer | CCA TTT CGT CGG GGT GTT CGT CCA | US20220064715A1 |
| | | TAC | PMID: 27807153 |
| GYRB2 out For | Primer | GCGGTCGCGATTCGATGTTCC | US20220064715A1 |
| | | | PMID: 27807153 |
| GYRB2 in For | Primer | GGCGATACTTCCGCTGCGCGGC | US20220064715A1 |
| | | | PMID: 27807153 |
| rpo1-probe | Probe | CGACCGCCCATAAATTGGCTCAGCTGGCT | US20200347439A1 |
| | | GGTGACGGTCG | PMID: 28851844 |
| | | | PMID: 22535987 |
| rpo2-probe | Probe | GGCGCGAACCACGACAGCGGGTTGTTCT | US20200347439A1 |
| | | GGTCCATGAACGCGCC | PMID: 28851844 |
| | | | PMID: 22535987 |
| rpo3-probe | Probe | CGCGGCCGACAGTTGGCGCTTGTGGGTT | US20200347439A1 |
| | | AACCCCGACGCCGCG | PMID: 28851844 |
| | | | PMID: 22535987 |
| rpoB-SMB4-probe | Probe | CGCGCGCCGGGCCCCAGCACCAACAGTC | US20220064715A1 |
| | | GGAGCTTCGCGCG | PMID: 28851844 |
| inhA-promoter-probe | Probe | ACC TGC CGC GGC GAG ACG ATA | US20220064715A1 |
| | | GGUTGT AGG GGT GAC GGC AGG T | PMID: 27807153 |
| katG-probe | Probe | CC GGC GAC ATC AAT GGT GCT GGT | US20220064715A1 |
| | | GAT CGC GTC CGC CGG | PMID: 27807153 |
| IS6110-probe | Probe | CGGCTGTGGGTAGCAGACCTCACC | US20220064715A1 |
| | | | PMID: 28851844 |
| IS1081-probe | Probe | CGCAGCGGTACTCGACGCTCTGACCGAC | US20220064715A1 |
| | | AAGCTGCG | PMID: 28851844 |
| gyrA1-probe | Probe | CCAGGGGATGGCGTAGATCGACGCGTCG | US20220064715A1 |
| | | CCGCGCCCTGG | PMID: 21191047 |
| | | | PMID: 27807153 |
| gyrA2-probe | Probe | CCAGGGGATGGCGTAGATCGGCACGTCG | US20220064715A1 |
| | | CCGCGCCCTGG | PMID: 21191047 |
| | | | PMID: 27807153 |
| gyrA3-probe | Probe | CCAGGGGATGCCGTAGATCGACACGTCG | US20220064715A1 |
| | | CCGCGCCCTGG | PMID: 21191047 |
| | | | PMID: 27807153 |
| gyrB2- probe | Probe | CCG AGC TGA TCG UCT GAA CTT CGG | US20220064715A1 |
| | | CGT UCT TTA GCA CCC GGT UGA TAG | PMID: 27807153 |
| | | CTC GG | |
| BG, FOR | Primer | AGC GCT TGC GGC AAA CAC GGA GAA A | PMID: 28851844 |
| | | | PMID: 27807153 |
| BG, REV1 | Primer | GCG ACA CCG GCG AAT ACA GAG ATA | PMID: 28851844 |
| | | CC | PMID: 27807153 |
| BG, REV2 | Primer | GCT CCA CCG AAC AAT CCG ATC TTA | PMID: 28851844 |
| | | CCG CCC | PMID: 27807153 |
| BG probe | Probe | CGGCCGACAGACAAGCT42CCGTCATTTG | PMID: 28851844 |
| | | AUCGGCC | |

The considerations for primers and probes for practicing the methods described herein are described in more detail below in the section entitled “Exemplary Polynucleotides.”

### Controls

In some embodiments, an assay described herein can include detecting at least one endogenous control in addition to the selected target nucleic acids. In some embodiments, the endogenous control is a Sample Adequacy Control (SAC). The SAC ensures that the sample contains human cells or human DNA. This assay includes primers and probes for the detection of a single-copy human gene. The SAC signal is only to be considered when the sample is negative for all other targets. A negative SAC indicates that no human cells are present in the sample due to insufficient mixing of the sample or because of an inadequately collected sample. In some such embodiments, if no target nucleic acid is detected in a sample, and the SAC is also not detected in the sample, the assay result is considered “invalid” because the sample may have been insufficient. While not intending to be bound by any particular theory, an insufficient sample may be too dilute, contain too little cellular material, or contain an assay inhibitor, etc. In some embodiments, the failure to detect an SAC may indicate that the assay reaction failed. In some embodiments, an endogenous control is an RNA (such as an mRNA, tRNA, ribosomal RNA, etc.). Nonlimiting exemplary endogenous controls include ABL mRNA, GUSB mRNA, GAPDH mRNA, TUBB mRNA, and UPKla mRNA.

In some embodiments, an assay described herein can include detecting the at least one exogenous control in addition to the selected target nucleic acids. In some embodiments, the exogenous control is a Sample Processing Control (SPC). The SPC verifies that sample processing is adequate. Additionally, this control detects sample-associated inhibition of the real-time PCR assay, ensures that the PCR reaction conditions (temperature and time) are appropriate for the amplification reaction, and that the PCR reagents are functional. The SPC should be positive in a negative sample and can be negative or positive in a positive sample. The SPC passes if it meets the validated acceptance criteria. In some such embodiments, if no target nucleic acid is detected in a sample, and the SPC is also not detected in the sample, the assay result is considered “invalid” because there may have been an error in sample processing, including but not limited to, failure of the assay. Nonlimiting exemplary errors in sample processing include, inadequate sample processing, the presence of an assay inhibitor, the presence of a nuclease (such as an RNase), or compromised reagents, etc. In some embodiments, an exogenous control (such as an SPC) is added to a sample. In some embodiments, an exogenous control (such as an SPC) is added during performance of an assay, such as with one or more buffers or reagents. In some embodiments, when a GENEXPERT® system is to be used, the SPC can be included in the GENEXPERT® cartridge. In some embodiments, an exogenous control (such as an SPC) is an Armored RNA®, which is protected by a bacteriophage coat.

In some embodiments, an endogenous control and/or an exogenous control is/are detected contemporaneously, such as in the same assay, as detection of the selected target nucleic acids. In some embodiments, an assay comprises reagents for detecting the target nucleic acids, and a SAC and/or an exogenous control, simultaneously in the same assay reaction mixture. In some such embodiments, for example, an assay reaction mixture comprises primer sets for amplifying the target nucleic acids, a primer set for amplifying a SAC and/or a primer set for amplifying an exogenous control, as well as optional labeled probes for detecting the amplification products (such as, for example, melt or TaqMan® probes).

In some embodiments, the assay includes a probe check control (PCC). In some such embodiments, before the start of the PCR reaction, the system (e.g., GENEXPERT System) measures the fluorescence signal from the probes to monitor bead rehydration, reaction tube filling, probe integrity, and dye stability. The PCC passes if it meets the validated acceptance criteria.

### Polynucleotides

In some embodiments, polynucleotides are provided for detecting the biomarkers described above. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot™ (GE Healthcare), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more analog of the canonical nucleotides (e.g., modified nucleotides).

In some embodiments, a polynucleotide is provided that comprises a region that is at least 85%, at least 90%, at least 91%, at least 92%, at least 93%, at least 94%, at least 95%, at least 96%, at least 97%, at least 98%, at least 99%, or 100% identical to, or at least 85%, at least 90%, at least 91%, at least 92%, at least 93%, at least 94%, at least 95%, at least 96%, at least 97%, at least 98%, at least 99%, or 100% complementary to, at least 6, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the selected target nucleic acids, and/or exemplary controls discussed above.

In various embodiments, an exemplary polynucleotide comprises at least: 5, 6, 7, 8, 9, 10, 11, 12, 13, 14, 15, 16, 17, 18, 19, 20, 21, 22, 23, 24, 25, 26, 27, 28, 29, or 30 nucleotides. In various embodiments, a polynucleotide comprises fewer than: 200, 150, 100, 50, 40, 30, or 20 nucleotides. In various embodiments, an exemplary polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, detection of each target nucleic acid can be carried out using a single labeled primer or probe, specific for each target nucleic acid. Different primers and/or probes can have the same label. By using primers or probes labeled with different detectable moieties (e.g., different fluorescent reporter dyes), numerous target nucleic acids can be detected simultaneously in a single reaction mixture. In some embodiments, 2, 3, 4, 5, 6, 7, 8, 9, 10, 11, 12, 13, 14, 15, 16, 17, 18, 19, or 20 or more different labels can be used in a single reaction mixture or a plurality of reaction mixtures. Each target nucleic acid can be independently monitored using such multiplexing technology. In some embodiments, detection of a plurality of target nucleic acids can be carried out using a single labeled primer or probe. A melt curve may be generated in order to distinguish two or more target nucleic acids that each use the same label, but such analysis may not be necessarily required.

### Polynucleotide Modifications

In some embodiments, the methods of detecting at least one target nucleic acid described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only the canonical deoxyribonucleotides, which allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications, and/or backbone modifications. In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine. In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester-modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy modifications, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14 (11): 1138-1142.

### Primers

In some embodiments, the polynucleotide is a primer. Primers useful in the methods described herein are generally capable of selectively hybridizing to: genomic DNA, a target RNA (genomic or transcript), a cDNA reverse transcribed from the target RNA, and/or an amplicon that has been amplified from genomic DNA, a target RNA, or a cDNA (collectively referred to as “template”), and, in the presence of the template, a polymerase and suitable buffers and reagents, can be extended to form a primer extension product. Primers are generally of a sufficient length to ensure selective hybridization to their target nucleic acids. Generally, primers of at least 15 nucleotides in length hybridize specifically in most contexts, and this length can be reduced, e.g., by including of affinity-enhancing modifications, such as those discussed above. Primers can but need not be exactly complementary to their target nucleic acids. Primers can have any degree of complementarity described above for exemplary polynucleotides. In illustrative embodiments, primers can be 8 to 40 nucleotides in length and at least 90% complementary to their target nucleic acids; 8 to 40 nucleotides in length and at least 95% complementary to their target nucleic acids; 8 to 40 nucleotides in length and at least 99% complementary to their target nucleic acids; 8 to 30 nucleotides in length and at least 90% complementary to their target nucleic acids; 8 to 30 nucleotides in length and at least 95% complementary to their target nucleic acids; 8 to 30 nucleotides in length and at least 99% complementary to their target nucleic acids. In embodiments wherein a primer is less than 100% complementary to it target nucleic acid, having 3′ nucleotide in the primer be complementary to its target nucleic acid facilitates the production of an extension product.

In some embodiments, a primer that selectively hybridizes to its target nucleic acid hybridizes to its target nucleic acid with at least 5-fold greater affinity than to non-target nucleic acid under the same assay conditions. In some embodiments, a primer that selectively hybridizes to its target nucleic acid hybridizes to its target nucleic acid with at least 10-fold greater affinity than to non-target nucleic acid under the same assay conditions.

In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

In some embodiments, the primer is labeled with a detectable moiety. In some embodiments, a primer is not labeled.

### Probes

In some embodiments, the polynucleotide is a probe. Probes useful in the methods described herein are generally capable of selectively hybridizing to: genomic DNA, a target RNA (genomic or transcript), a cDNA reverse transcribed from the target RNA, and/or an amplicon that has been amplified from genomic DNA, a target RNA, or a cDNA (collectively referred to as “template”). Generally, probes of at least 15 nucleotides in length hybridize specifically in most contexts, and this length can be reduced, e.g., by including of affinity-enhancing modifications, such as those discussed above. Probes can but need not be exactly complementary to their target nucleic acids. Probes can have any degree of complementarity described above for exemplary polynucleotides. In illustrative embodiments, probes can be 8 to 40 nucleotides in length and at least 90% complementary to their target nucleic acids; 8 to 40 nucleotides in length and at least 95% complementary to their target nucleic acids; 8 to 40 nucleotides in length and at least 99% complementary to their target nucleic acids; 8 to 30 nucleotides in length and at least 90% complementary to their target nucleic acids; 8 to 30 nucleotides in length and at least 95% complementary to their target nucleic acids; 8 to 30 nucleotides in length and at least 99% complementary to their target nucleic acids. In embodiments wherein a primer is less than 100% complementary to a target nucleic acid, any points or regions of non-complementarity are typically located so as not to disrupt the ability of the probe to selectively hybridize to its target nucleic acid.

In some embodiments, a probe that selectively hybridizes to its target nucleic acid hybridizes to its target nucleic acid with at least 5-fold greater affinity than to non-target nucleic acid under the same assay conditions. In some embodiments, a probe that selectively hybridizes to its target nucleic acid hybridizes to its target nucleic acid with at least 10-fold greater affinity than to non-target nucleic acid under the same assay conditions.

### Polynucleotide Labels

In some embodiments, the primer or probe is labeled with a detectable moiety. Detectable moieties include directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a primer or probe is not labeled, such as when a primer or probe is immobilized, e.g., on a microarray or bead. A labeled primer is extendable, e.g., by a polymerase. In some embodiments, a probe is extendable. In other embodiments, a probe is not extendable. The following discussion centers on probes, as these are more typically employed for detecting in the methods described here, but those of skill in the art appreciate that the polynucleotide labeling strategies described below apply equally to the labeling of primers.

In some embodiments, the probe is a FRET probe that, in some embodiments, is labeled at 5′-end with a fluorescent dye (donor) and at 3′-end with a quencher (acceptor), a chemical group that absorbs (i.e., suppresses) fluorescence emission from the dye when the groups are in close proximity (e.g., attached to the same probe). Thus, in some embodiments, the emission spectrum of the dye should overlap considerably with the absorption spectrum of the quencher. In other embodiments, the dye and quencher are not at the ends of the FRET probe.

Illustrative FRET probes, which include, but are not limited to, a TaqMan® probe, a Molecular beacon probe and a Scorpion probe. A TaqMan® probe is a linear probe that typically has a fluorescent dye covalently bound at one end of the DNA and a quencher molecule covalently bound elsewhere, such as at the other end of the DNA. The FRET probe comprises a sequence that is complementary to a region of the cDNA or amplicon such that, when the FRET probe is hybridized to the cDNA or amplicon, the dye fluorescence is quenched, and when the probe is digested during amplification of the cDNA or amplicon, the dye is released from the probe and produces a fluorescence signal. In some embodiments, the amount of target nucleic in the sample is proportional to the amount of fluorescence measured during amplification.

Like TaqMan® probes, Molecular Beacons use FRET to detect a PCR product via a probe having a fluorescent dye and a quencher attached at the ends of the probe. Unlike TaqMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target nucleic acid, the stem-loop structure is abolished so that the dye and the quencher become separated in space and the dye fluoresces. Molecular Beacons are available, e.g., from Gene Link™ (see www.genelink.com/newsite/products/mbintro.asp).

In some embodiments, Scorpion probes can be used as sequence-specific primers and for PCR product detection. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to 5′-end of the Scorpion probe, and a quencher is attached elsewhere, such as to 3′-end. The 3′ portion of the probe is complementary to the extension product of the PCR primer, and this complementary portion is linked to 5′-end of the probe by a non-amplifiable moiety. After the Scorpion primer is extended, the target-specific sequence of the probe binds to its complement within the extended amplicon, thus opening up the stem-loop structure and allowing the dye on 5′-end to fluoresce and generate a signal. Scorpion probes are available from, e.g., Premier Biosoft International (see www.premierbiosoft.com/tech_notes/Scorpion.html).

In some embodiments, labels that can be used on the FRET probes include colorimetric and fluorescent dyes, such as Alexa Fluor dyes; BODIPY dyes, such as BODIPY FL, Cascade Blue, and Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; and fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, IA) or a Black Hole Quencher™ (BHQ™; Sigma-Aldrich, St. Louis, MO).

Specific examples of fluorescently labeled ribonucleotides useful in the preparation of probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Specific examples of fluorescently labeled deoxyribonucleotides useful in the preparation of probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, and Alexa Fluor 647-12-OBEA-dCTP. Fluorescently labeled nucleotides are commercially available and can be purchased from, e.g., Invitrogen.

As noted above, exemplary detectable moieties also include members of binding pairs. Exemplary binding pairs include, but are not limited to, biotin and streptavidin, antibodies and antigens, etc.

Single PCR reactions (nucleic acid amplification), or multiple PCR reactions (nucleic acid amplifications and/or melt analysis) run sequentially (or simultaneously in separate temperature controlled channels or chambers) can use the same detectable label since sequentially run PCR signal DNAs are analyzed sequentially and the simultaneous PCR signal DNAs are distinguished by the occurrence in different temperature controlled channels or chambers. The signal produced by this amplification can be distinguished from other amplification products because it is not run at the same time and/or because it is run in a different reaction channel/chamber. However, where multiple nucleic acid amplifications are run simultaneously in the same chamber, the reaction products of for each analysis are typically detected and/or quantified by the use of different and distinguishable labels.

In certain embodiments, amplification products (amplified nucleic acid from nucleic acid analysis) can be detected using methods well known to those of skill in the art. In certain embodiments the amplification is a straightforward simple PCR amplification reaction. In certain embodiments, however, a nested PCR reaction is used to amplify the nucleic acid from the nucleic acid analysis. In various embodiments, multiplexed PCR assays are contemplated, particularly where it is desired to analyze multiple products of the nucleic acid analysis in the same amplification reaction. In certain embodiments in such multiplexed amplification reactions, each probe (e.g., for each specific analyte) has its own specific dye/fluor so that it is detectable independently of the other probes. In certain embodiments, typically, for signal generation, the probes used in various amplification reactions utilize a change in the fluorescence of a fluorophore due to a change in its interaction with another molecule or moiety brought about by changing the distance between the fluorophore and the interacting molecule or moiety for detection and/or quantification of the amplified product. Alternatively, other methods of detecting a polynucleotide in a sample, including, but not limited to, the use of radioactively labeled probes, are contemplated.

### Sample

The sample to be tested can be any sample suspected of containing the target nucleic acids. In some embodiments, the sample is a biological sample collected from a subject. In other embodiments, the sample is a sample that is not collected directly from a subject, such as, e.g., a wastewater sample or a sample from an air filter in a building.

Illustrative biological samples include the following: a sputum sample, a nasal aspirate sample, a nasal wash sample, a nasal swab sample, a nasopharyngeal swab sample, a saliva sample, an oropharyngeal swab sample, a throat swab sample, a bronchoalveolar lavage sample, a bronchial aspirate sample, a bronchial wash sample, an endotracheal aspirate sample, an endotracheal wash sample, a tracheal aspirate sample, a nasal secretion sample, a mucus sample, a pleural effusion sample, a cerebrospinal fluid sample, a stool sample, a tissue biopsy sample, a breath sample, or a combination thereof.

The sample to be tested is, in some embodiments, fresh (i.e., never frozen). In other embodiments, the sample is a frozen specimen. In some embodiments, the sample is a tissue sample, such as a formalin-fixed paraffin embedded sample. In some embodiments, the sample is a liquid cytology sample. In some embodiments, e.g., assays for resistance to tuberculosis drugs, the sample can be a sputum sample that is unprocessed or decontaminated, digested, and concentrated.

In some embodiments, a sample to be tested is contacted with a buffer after collection. For example, in the case of a sputum sample, a buffer (including, e.g., a preservative) can be added to the sample. In embodiments where the sample is a swab sample, the swab can simply be placed in a buffer. In some embodiments, that sample is contacted with the buffer immediately; in the case of a swab, the swab is immediately placed in the buffer. In some embodiments, the sample (e.g., including the swab) is contacted with buffer within 5 minutes, within 10 minutes, within 30 minutes, within 1 hour, or within 2 hours of sample collection.

In some embodiments, less than 5 ml, less than 4 ml, less than 3 ml, less than 2 ml, less than 1 ml, or less than 0.75 ml of sample or buffered sample are used in the present methods. In some embodiments, 0.1 ml to 1 ml of sample or buffered sample is used in the present methods.

### Subjects

A biological sample useful in the methods described herein can be collected from any subject that may have one or more of the target nucleic acids. In various embodiments, the subject can include non-human animals, e.g., canines, felines, equines, primates, and other non-human mammals, as well as humans.

In some embodiments, the sample to be tested is obtained from an individual who has one or more symptoms of tuberculosis or who has been exposed to such an individual. The primary symptoms of tuberculosis include persistent cough, cough with blood in the sputum, fever, and chest pain.

In some embodiments, methods described herein can be used for routine screening of apparently healthy individuals with no risk factors. In some embodiments, methods described herein are used to screen asymptomatic individuals, for example, during routine or preventative care. In some embodiments, methods described herein are used to screen women who are pregnant or who are attempting to become pregnant.

In some embodiments, the methods described herein can be used to assess the effectiveness of a treatment in an individual undergoing treatment e.g., for tuberculosis.

### Assay Methods

Any analytical procedure capable of permitting specific detection of a target nucleic acid can be used in the methods herein presented. In some embodiments, DNA targets can be detected by direct hybridization or, more easily, by amplification of the DNA template and detection of the amplicon. In some embodiments, RNA targets can be detected by direct hybridization or, more easily, by reverse transcribing a target RNA to produce a cDNA that is complementary to the target RNA. This cDNA can be directly detected by direct hybridization or by amplification of the cDNA template.

Nucleic acid amplification provides rapid, sensitive, and specific detection of nucleic acid targets, and has been employed in a wide variety of assay formats to detect nucleic acid targets. Those of skill in the art can, following the guidance herein, carry out the methods described herein in any number of different nucleic acid amplification-based assays, using, for example, any of the nucleic acid amplification methods discussed above. Such methods can entail thermocycling, but need not do so, as in the case of isothermal amplification. Exemplary methods include, but are not limited to, isothermal amplification, real-time RT-PCR, endpoint RT-PCR, and amplification using T7 polymerase from a T7 promoter annealed to a DNA, such as provided by the SenseAmp Plus™ Kit available at Implen, Germany. Amplification and detection can be carried out in solution or can make use of a solid support (e.g., a biochip). Nucleic acid amplification-based assays can employ a single reaction chamber or multiple reaction chambers. Amplification can be nested or non-nested. In some embodiments, detection includes electrochemical detection.

In some embodiments, target nucleic acids, and/or optional controls, can be detected by (a) contacting nucleic acid from the sample with a set of primers and optional probes for detecting the presence of the desired target nucleic acids, (b) subjecting the nucleic acid, primers, and optional probes to amplification conditions; (c) detecting the presence of any amplification product(s), optionally via real-time PCR, melt curve analysis, or a combination thereof, and (d) differentially identifying the presence of a viral pathogen in the sample, or determining that no viral pathogen detectable using the set of primers is present, based on detection of the amplification product(s) or lack thereof, respectively. In this context, “differentially identifying” refers to the ability to determine that a particular target organism is present and that one or more other target organisms of the assay are not. In some embodiments, the assay is able to determine the presence of any target organism this present in the sample, while ruling out the presence of the other target organisms (above the detection limit of the assay).

In some embodiments of amplification by polymerase chain reaction (PCR), an exemplary cycle comprises an initial denaturation at 90° C. to 100° C. for 20 seconds to 5 minutes, followed by cycling that comprises denaturation at 90° C. to 100° C. for 1 to 10 seconds, followed by annealing and amplification at 60° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot-start function. In some embodiments, detection of the target nucleic acids occurs in less than 3 hours, less than 2.5 hours, less than 2 hours, less than 1 hour, or less than 30 minutes from initial denaturation through the last extension. In some embodiments, target nucleic acids are detected by a method that includes real-time quantitative PCR, e.g., using FRET probes, such as those described above.

In some embodiments, quantitation of the results of real-time PCR assays is done by constructing a standard curve from a nucleic acid of known concentration and then extrapolating quantitative information for target nucleic acids of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to indicate that a given target nucleic acid is not present in a sample, the Ct values for an endogenous control (such as an SAC) and/or an exogenous control (such as an SPC) must be within previously-determined valid ranges. For example, in some embodiments, the absence of a particular target nucleic acid cannot be confirmed unless the controls are detected, indicating that the assay was successful.

In some embodiments, a threshold Ct (or a “cutoff Ct”) value for a target nucleic acid (including an endogenous control and/or exogenous control), below which the gene is considered to be detected, has previously been determined. In some embodiments, a threshold Ct is determined using substantially the same assay conditions and system (such as a GENEXPERT®) on which the samples will be tested.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, the number of target nucleic acids in an assay exceeds the number of labels that can be detected, e.g., in a particular instrument. Therefore, the PCR amplification can be followed by a melt analysis to increase the number of possible reported results. In general, target organisms requiring high sensitivity can be detected with real-time PCR detection using TaqMan probes or molecular beacon probes and/or detected with melt analysis.

Another approach to detect target nucleic acids can include high-resolution melt alone. Endpoint melting curve data to detect target nucleic acids and analyses can be performed to generate a result for each analyte.

Target nucleic acids can also be detected by real-time PCR but in more than one reaction chambers. Another approach to detect a target nucleic acid can include digital microfluidics or electrowetting and electrochemical detection. For example, digital microfluidics or electrowetting, responsible for the movement and transfer of samples and reagents inside a cartridge can be conducted. Systems for such can include a microarray for detection, consisting of target-specific capture probes attached to gold electrodes (solid-support), which generates a voltage signal if a “target DNA/signal probe” hybridizes with the capture probes. Target nucleic acids can also be detected using a chip that includes an integrated sensor array.

Examples of other approaches that can be employed in the methods describe herein include bead-based flow cytometric assay. See Lu J. et al. (2005) Nature 435:834-838, which is incorporated herein by reference for this description. An example of a bead-based flow cytometric assay is the xMAP® technology of Luminex, Inc. See www.luminexcorp.com/technology/index.html. Another approach uses microfluidic devices and single-molecule detection. See U.S. Pat. Nos. 7,402,422 and 7,351,538 to Fuchs et al, U.S. Genomics, Inc., each of which is incorporated herein by reference in its entirety. Yet another approach is simple gel electrophoresis and detection with labeled probes (e.g., probes labeled with a radioactive or chemiluminescent label), such as by northern blotting.

In some embodiments, the approach for detecting a target nucleic acid does not include bead-based flow cytometric assay, microfluidic devices and single-molecule detection, simple gel electrophoresis, use of a capture probe attached to a solid-support, separation of reaction mixture into multiple reaction chambers, array-based detection, nested amplification, electrochemical detection, high resolution melt only, or a combination thereof.

### Automated Assay Methods

Readily automated approaches are of great interest. The methods described herein can be carried out in a substantially automated manner using a commercially available nucleic acid amplification system. Exemplary nonlimiting nucleic acid amplification systems that can be used to carry out the methods of the invention include the GENEXPERT® system, a GENEXPERT® Infinity system, and GENEXPERT® Xpress System (Cepheid, Sunnyvale, Calif.). In some embodiments, the amplification system may be available at the same location as the individual to be tested, such as a health care provider's office, a clinic, or a community hospital, so processing is not delayed by transporting the sample to another facility. Assays according to the method described herein can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system. The GENEXPERT® utilizes a self-contained, single-use cartridge. Sample extraction, amplification, and detection may all carried out within this self-contained sample cartridge as described herein.

In some embodiments, after the sample is added to the cartridge, the sample is contacted with lysis buffer and released nucleic acid (NA) is bound to a NA-binding substrate, such as a silica or glass substrate. The sample supernatant is then removed and the NA eluted in an elution buffer, such as a Tris/EDTA buffer. The eluate may then be processed in the cartridge to detect target nucleic acids as described herein. In some embodiments, the eluate is used to reconstitute at least some of the PCR reagents, which are present in the cartridge as lyophilized particles.

A cartridge having a plurality of chambers can have the set of primers and optional probes described herein, or a subset thereof, disposed in a chamber. In some embodiments, the set of primers and optional probes described herein, or a subset thereof, are disposed in more than one of the plurality of chambers.

In some embodiments, RT-PCR is used to amplify and analyze the presence of the target nucleic acids. In some embodiments, the reverse transcription uses MMLV and/or CAT-A RT enzyme and an incubation of 5 to 20 minutes at 40° C. to 50° C. In some embodiments, the PCR uses Taq polymerase with hot-start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed.

In some embodiments, a double-denature method is used to amplify low-copy number target nucleic acids. A double-denature method comprises, in some embodiments, a first denaturation step followed by addition of primers and/or probes for detecting target nucleic acids. All or a substantial portion of the nucleic acid-containing sample (such as a DNA eluate) is then denatured a second time before, in some instances, a portion of the sample is aliquoted for cycling and detection of the target nucleic acids. While not intending to be bound by any particular theory, the double-denature protocol may increase the chances that a low-copy number target nucleic acid (or its complement) will be present in the aliquot selected for cycling and detection because the second denaturation effectively doubles the number of target nucleic acids (i.e., it separates the target nucleic acid and its complement into two separate templates) before an aliquot is selected for cycling. In some embodiments, the first denaturation step comprises heating to a temperature of 90° C. to 100° C. for a total time of 30 seconds to 5 minutes. In some embodiments, the second denaturation step comprises heating to a temperature of 90° C. to 100° C. for a total time of 5 seconds to 3 minutes. In some embodiments, the first denaturation step and/or the second denaturation step is carried out by heating aliquots of the sample separately. In some embodiments, each aliquot may be heated for the times listed above. As a non-limiting example, a first denaturation step for an NA-containing sample (such as a DNA eluate) may comprise heating at least one, at least two, at least three, or at least four aliquots of the sample separately (either sequentially or simultaneously) to a temperature of 90° C. to 100° C. for 60 seconds each. As a non-limiting example, a second denaturation step for a NA-containing sample (such as a DNA eluate) containing enzyme, primers, and probes may comprise heating at least one, at least two, at least three, or at least four aliquots of the eluate separately (either sequentially or simultaneously) to a temperature of 90° C. to 100° C. for 5 seconds each. In some embodiments, an aliquot is the entire NA-containing sample (such as a DNA eluate). In some embodiments, an aliquot is less than the entire NA-containing sample (such as a DNA eluate).

In some embodiments, an off-line centrifugation is used, for example, with samples with low cellular content. The sample, with or without a buffer added, is centrifuged and the supernatant removed. The pellet is then resuspended in a smaller volume of either supernatant or the buffer. The resuspended pellet is then analyzed as described herein.

### Exemplary Automation and Systems

Many existing fully integrated nucleic acid amplification and test systems capable of sample preparation are normally quite complicated and costly. The nucleic acid amplification and test systems provided herein perform rapid, simple, convenient, and affordable nucleic acid analysis.

### System Overview

In one aspect, the invention pertains to a sample cartridge that utilizes a valve body platform that allows for detection of enveloped and free target nucleic acids. In some embodiments, the valve body includes a sample processing region or lysing chamber that provides for either or both mechanical and chemical lysis. This allows a single cartridge to provide lysing for a multitude of differing types of targets, thus, can be considered a “panel assay cartridge.” In some embodiments, the sample cartridge can perform processing and detection of viral targets suited for chemical lysing.

The sample cartridge device can be any device configured to perform one or more process steps relating to preparation and/or analysis of a biological fluid sample according to any of the methods described herein. In some embodiments, the sample cartridge device is configured to perform at least sample preparation. The sample cartridge can further be configured to perform additional processes, such as detection of a target nucleic acid in a nucleic acid amplification test (NAAT), e.g., Polymerase Chain Reaction (PCR) assay, by use of a reaction vessel attached to the sample cartridge. In some embodiments, the reaction vessel extends from the body of the cartridge. Preparation of a fluid sample generally involves a series of processing steps, which can include chemical, electrical, mechanical, thermal, optical or acoustical processing steps according to a specific protocol. Such steps can be used to perform various sample preparation functions, such as cell capture, cell lysis, binding of analyte, and binding of unwanted material.

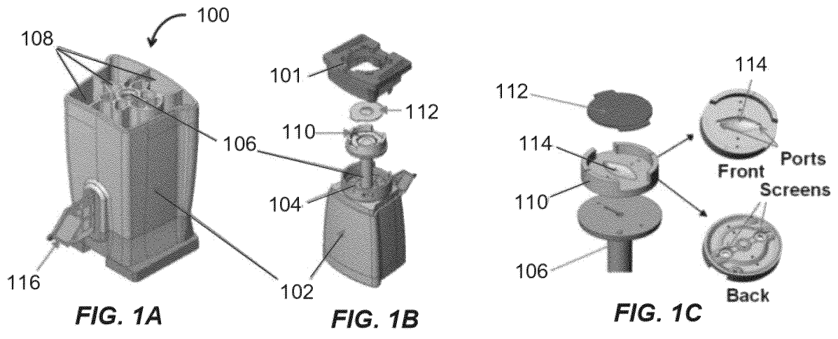

A sample cartridge suitable for use with the invention, includes one or more transfer ports through which the prepared fluid sample can be transported into an attached reaction vessel for analysis. FIG. 1A illustrates an exemplary assay cartridge 100 suitable for sample preparation and analytics testing by PCR when received in an instrument module in accordance with some embodiments. The sample cartridge is attached with a reaction vessel 116 (also referred to as a “reaction tube” or “PCR tube”) adapted for analysis of a fluid sample processed within the sample cartridge 100. In some embodiments the reaction vessel extends from the cartridge body. Such a sample cartridge 100 includes various components including a main housing 102 having one or more chambers 108 for processing of the fluid sample, which typically include sample preparation before analysis. In these embodiments, the sample cartridge can be a fully integrated nucleic acid amplification and test system combining sample preparation, amplification, and detection together. The instrument module facilitates the processing steps needed to perform sample preparation and the prepared sample is transported through one of a pair of transfer ports into fluid conduit of the reaction vessel 116 attached to the housing of the sample cartridge 100. The prepared biological fluid sample is then transported into a reaction chamber of the reaction vessel where the biological fluid sample undergoes nucleic acid amplification. In some embodiments, the amplification is a polymerase chain reaction. In some embodiments, concurrent with the amplification of the biological fluid sample, an excitation means, and an optical detection means of the module is used to detect optical emissions that indicate the presence or absence of a target nucleic acid analyte of interest, e.g., a bacterium, a virus, a pathogen, a toxin, or other target analyte. It is appreciated that such a reaction vessel could include various differing chambers, conduits, or micro-well arrays for use in detecting the target analyte. The sample cartridge can be provided with means to perform preparation of the biological fluid sample before transport into the reaction vessel. Any chemical reagent required for viral or cell lysis or means for binding or detecting an analyte of interest (e.g., reagent beads) can be contained within one or more chambers of the sample cartridge, and as such can be used for sample preparation.

An exemplary use of a reaction vessel for analyzing a biological fluid sample is described in commonly assigned U.S. Pat. No. 6,818,185, entitled “Cartridge for Conducting a Chemical Reaction,” filed May 30, 2000, the entire contents of which are incorporated herein by reference for all purposes. Examples of the sample cartridge and associated modules are shown and described in U.S. Pat. No. 6,374,684, entitled “Fluid Control and Processing System” filed Aug. 25, 2000, and U.S. Pat. No. 8,048,386, entitled “Fluid Processing and Control,” filed Feb. 25, 2002, U.S. Patent Application No. 63/217,672 entitled “Universal Assay Cartridge and Methods of Use” filed Jul. 1, 2021; U.S. Provisional Application No. 63/319,993 entitled “Unitary Cartridge Body and Associated Components and Methods of Manufacture” filed Mar. 15, 2022; and U.S. Pat. No. 10,562,030 entitled “Molecular Diagnostic Assay System” filed Jul. 22, 2016; the entire contents of which are incorporated herein by reference in their entirety for all purposes.

Various aspects of the sample cartridge 100 can be further understood by referring to U.S. Pat. No. 6,374,684 “the '684 patent”), which described certain aspects of a sample cartridge in greater detail. Such sample cartridges can include a fluid control mechanism, such as a rotary fluid control valve assembly, that is fluidically connected to the chambers of the sample cartridge. The term “chamber” can be used interchangeably with the terms “well”, “tube”, and the like. Rotation of the rotary fluid control valve permits fluidic communication between chambers and the valve so as to control flow of a biological fluid sample deposited in the cartridge into different chambers in which various reagents can be provided according to a particular protocol as needed to prepare the biological fluid sample for analysis. To operate the rotary valve, the cartridge processing module comprises a motor such as a stepper motor that is typically coupled to a drive train that engages with a feature of the valve in the sample cartridge to control movement of the valve in coordination with movement of the syringe, thereby resulting movement of the fluid sample according to the desired sample preparation protocol. The fluid metering and distribution function of the rotary valve according to a particular sample preparation protocol is demonstrated in the '684 patent.

### Exemplary Assay Cartridge

As shown in FIG. 1A, the assay cartridge 100 comprises a cartridge body 102 containing a plurality of chambers 108 for reagents or buffers and sample processing. The chambers are disposed around a central syringe barrel 106 that is in fluid communication with a valve body 110 (see FIGS. 1B and 1C) and that is sealed with a gasket 104. The valve body 110 can include a cap 112 and the entire cartridge body can be supported on a cartridge base 101. The valve body typically contains one or channels or cavities (chamber(s) 114) that can contain a filter as described herein that can function to bind and elute a nucleic acid. In some embodiments the cartridge further comprises one or more temperature-controlled channels or chambers that can, in certain embodiments, function as thermocycling chambers. A “plunger” not shown can be operated to draw fluid into the syringe barrel 106 and rotation of the valve body 110 provides selective fluid communication between the various reagent chambers 108 and channels, reaction chamber(s), mixing chambers, and optionally, any temperature controlled regions. Thus, the various reagent chambers 108, reaction chambers, filter material(s), and temperature-controlled chambers or channels are selectively in fluid communication by rotation of the plunger and reagent movement (e.g., chamber loading or unloading) is operated by the “syringe” action of the plunger within the valve assembly. In other embodiments, the various reagent chambers, reaction chambers, filter material, and temperature-controlled chambers or channels are selectively in fluid communication by linear progression (e.g., by forced movement) of the reagents and sample from one chamber to the next.

### Reaction Modules

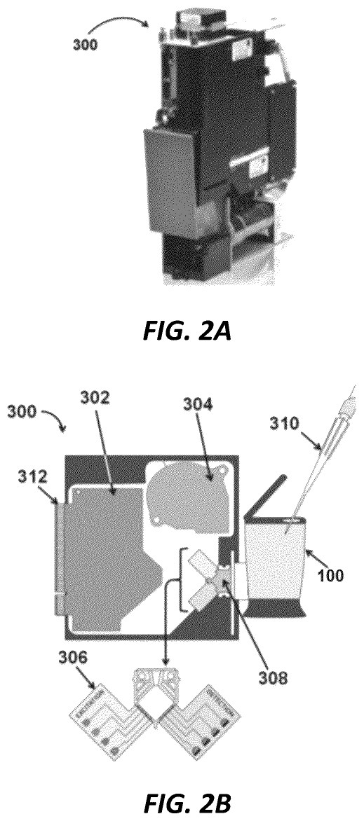

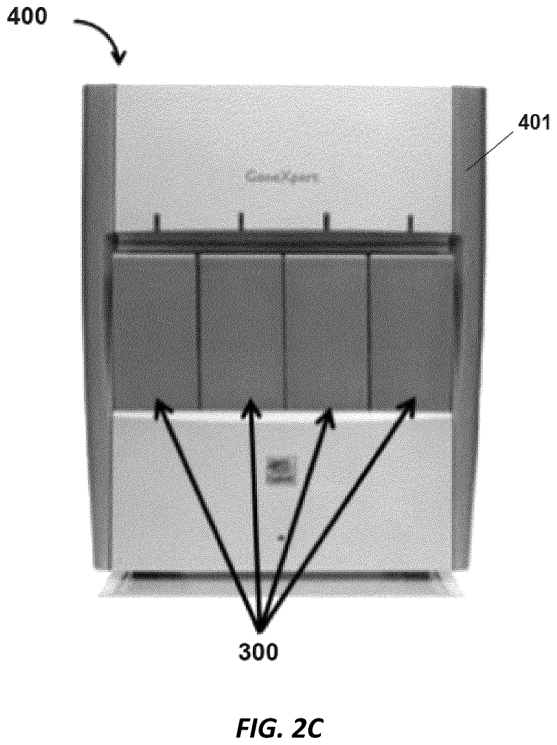

In certain embodiments the cartridge 100 is configured for insertion into a reaction module 300, e.g., as shown in FIG. 2A. As illustrated in FIG. 2B the module is configured to receive the cartridge 100 therein. In certain embodiments the reaction module provides heating plates 308 to heat the temperature controlled chamber or channel. The module can optionally additionally include a fan 304 to provide cooling where the temperature controlled channel or chamber is a thermocycling channel or chamber. Electronic circuitry 302 can be provided to pass information (e.g., optical information) top a computer for analysis. In certain embodiments the module can contain optical blocks 306 to provide excitation and/or detection of one or more (e.g., 1, 2, 3, 4, 5, 6, 7, 8, 9, 10, or more) optical signals representing, e.g., signal DNAs amplified for various PCR targets. In various embodiments an electrical connector 312 can be provided for interfacing the module with a system (e.g. system controller or with a discrete analysis/controller unit). As illustrated, in FIG. 2B the sample can be introduced into the cartridge using a pipette 310. In certain embodiments, the module also contains a controller that operates a plunger in the syringe barrel and the rotation of the valve body.

While the methods described herein are described primarily with reference to the GENEXPERT® cartridge by Cepheid Inc. (Sunnyvale, Calif.) (see, e.g., FIG. 1A), it will be recognized, that in view of the teachings provided herein the methods can be implemented on other cartridge/microfluidic systems, including alternative cartridge designs having valve assemblies that involve multiple interfacing components, as well as cartridge body defined by multiple interfacing components to form the multiple chambers of the cartridges, for example, those described in Korean Application No. 102293717B1 and KR102362853B1, cartridges that utilizes ultrasonic waves to lyse cells in a biological sample, for example, those described in International Application No. WO2021/245390A1, cartridges and systems that utilizes an electrowetting grid for microdroplet manipulation and electrosensor arrays configured to detect analytes of interest, for example, those described in International Application No. WO2016/077341A2, cartridges that facilitate movement of nucleic acid from one chamber to the next chamber by opening a vent pocket, for example, those described in International Application No. WO2012/145730A2, multiplexed assay systems comprising a plurality of thermocycling units such that individual chambers can be heated, cooled, and/or compressed to mix fluid within the chamber or to propel fluid in the chamber into another chamber, for example, those described in International Application No. WO2015/138343A1, and as well as systems for rapid amplification of nucleic acids facilitated by flexible portions of the sample cartridge aligned to accomplish temperature cycling for nucleic acid amplification, for example, those described in International Application No. WO2017/147085A1. Such cartridge/microfluidic systems can include, for example microfluidic systems implemented using soft lithography, micro/nano-fabricated microfluidic systems implemented using hard lithography, and the like.

In an exemplary embodiment, the cartridge can include a plurality of reaction chambers, particularly, the reaction vessel can include a plurality of reaction chambers. In these embodiments, different types of lyophilized primers and probes can be provided in each reaction chamber. For example, primers and probes for viral-associated nucleic acids can be provided in one reaction chamber, and primers and probes for viral-associated nucleic acids can be provided in a second chamber for amplification and detection, and such the like. Of course, it is possible to perform various amplification and detection processes at the same time in a single reaction chamber. Accordingly, amplification of each target nucleic acid described herein may be performed individually in separate reaction chambers or wells or carried out in a multiplex reaction in a single reaction chamber or well.

Additionally, it is appreciated that the panel assay methods described herein (i.e., identification of multiple conditions based on comparative levels of multiple-target nucleic acids obtained from a single sample) can further be realized in entirely different systems, including: gradient PCR, isothermal nucleic acid amplification systems, digital RT-PCR, electrochemical PCR, lateral flow testing cartridges, electrochemical sensors, nucleic acid sequencing, CRISPR/Cas based technologies, chemiluminescence, and nanoparticle-based colorimetric detection.

In various embodiments, the signal DNA(s) from PCR (nucleic acid amplification) reactions are amplified for detection and/or quantification. In certain embodiments, the amplification comprise any of a number of methods including, but not limited to polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), rolling circle amplification (RCA), and the like.

### Assay Workflows

Prior to carrying out amplification reactions on a sample, one or more sample preparation operations are performed on the sample. Typically, these sample preparation operations will include such manipulations as extraction of intracellular material, e.g., nucleic acids from whole cell samples, viruses and the like to form a crude extract, additional treatments to prepare the sample for subsequent operations, e.g., denaturation of contaminating (e.g., DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced. Preferably, sample preparation is carried out in only one step or no more than two steps. For example, sample preparation can include heating the sample in a lysis solution without further purification prior to carrying out the amplification reaction. In some embodiments, the lysed sample may be diluted prior to carrying out the amplification reaction. One or more of these various sample preparation operations are readily incorporated into the fluidly closed cartridge systems contemplated herein.

In one aspect, the assay sample cartridge, as described herein, is capable of a specialized workflow that performs lysing and detection of differing target analytes as required for a particular panel assay. In some embodiments, the cartridge is configured for chemical lysing of the multiple target organisms. In other embodiments, the cartridge is configured for mechanical lysing of the multiple target organisms. In still other embodiments, the cartridge is configured for both mechanical and chemical lysing to allow lysing of multiple targest of differing types. Accordingly, the sample cartridge can be configured to perform the panel assay by an existing workflow associated with conventional cartridges, or can be operated according to an new workflow specially configured for the panel assay.

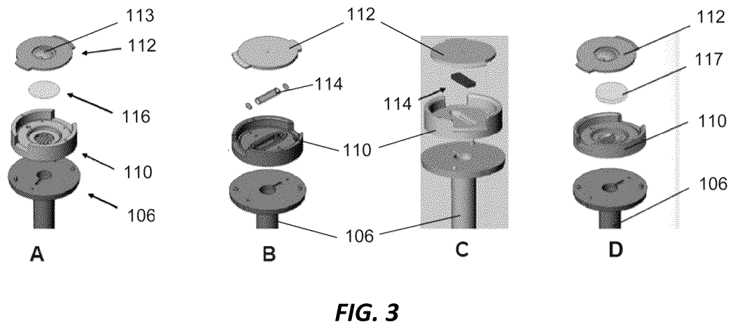

In one aspect, the sample cartridge having a valve assembly as described in FIG. 3D herein, is capable of a variety of workflows that perform: chemical lysing of target organisms, mechanical lysing of target organisms, or both. Accordingly, the sample cartridge can perform an existing workflow associated with conventional specialized cartridges or can perform entirely new workflows that perform both.

Exemplary assay workflows A-C (below) can be performed with a single universal cartridge. In any of these embodiments, the filter can be formed of glass filter to promote affinity binding of the nucleic acids (NA) to the glass fibers and a pore size suited for chemical lysing as well. In any of these workflows, the nucleic acid amplification can be PCR, real-time PCR, isothermal amplification (including but not limited to nucleic acid sequence-based amplification, loop-mediated isothermal amplification, helicase-dependent amplification, rolling circle amplification, multiple displacement amplification, whole genome amplification or recombinase polymerase amplification) or other nucleic acid amplification methods known to persons of skill in the art.

In Workflow A, the sample is optionally exposed to a sample treatment or chemically lysed, then the treated or lysed fluid sample is flowed through the filter where target organisms are captured. In some embodiments, the sample treatment is used to either weaken the cell wall or to inactivate the sample or make it less viscous to facilitate being processed through the filter. The filter is then washed, leaving the target organisms on the filter. Next, the target organisms are mechanically lysed, such as by sonication, to release nucleic acid (NA). In some embodiments, mechanical lysing includes in-filling glass beads along the filter to aid in mechanical lysing of the target. Next, the NA is eluted from the filter and then nucleic acid amplification is performed is performed.

In Workflow B, the sample is chemically lysed to obtain the NA targets. In some embodiments, after chemically lysing, the NA is bound to the filter by the presence of precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the filter is eluted to remove the NA targets. In some embodiments, the elution is performed with a pH neutral buffer or basic buffer fluid. The target NA is then delivered to an attached reaction vessel to perform nucleic acid amplification.

In Workflow C, the fluid sample is exposed to sample treatment and/or chemically lyse the target organisms. Next, the NA freed by chemical lysing is bound to the filter. This step may utilize precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the target organisms captured in the filter are heat and/or mechanically lysed. This may utilize sonication, and may further utilize glass beads to facilitate mechanical lysing of select target organisms. Then, the lysed target NA is eluted from the filter. In some embodiments, the elution is performed with a pH neutral buffer or basic buffer fluid. The target NA is then delivered to an attached reaction vessel to perform nucleic acid amplification. Thus, in this workflow, the workflow allows for lysing of multiple differing target organisms, some requiring only chemically lysing (e.g. viral targets), and others requiring mechanical lysing (e.g. bacteria, spores, etc.), such that all these target NAs can be released from a single sample and tested by the same sample cartridge. While the above workflow described mechanical lysing after chemical lysing, it is appreciated that other workflows may be utilized in which chemical lysing occurs after mechanical lysing.

In some embodiments, the sample cartridge includes an identifier with information as to the appropriate workflow needed for a particular panel of assays, so that an instrument module receiving the sample cartridge operates according to the specified workflow.

### Exemplary Assay Configurations

### Reagents

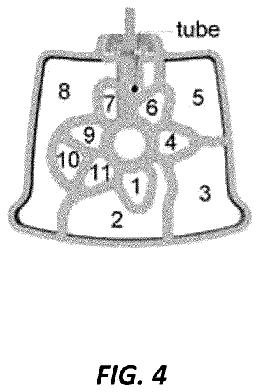

FIG. 4 shows an exemplary sample cartridge than can be used as part of the GENEXPERT® system, with the various chambers designated by number. The exemplary cartridge can include: lyophilized reagents in the form of one or more beads as described herein; an optional lysis reagent; alkaline agent, optional binding reagent, filtering reagent, washing reagent, and eluting reagent. The last four reagents are named according to the functions they perform with respect to nucleic acid. Thus, for example the binding reagent facilitates the binding of nucleic acid to a substrate, the filtering reagent facilitates filtration of the nucleic acid.

In some embodiments, the lysis reagent can include a chaotropic agent, a chelating agent, a buffer, an alkaline agent, or a detergent. The chaotropic agent can be selected from a guanidinium compound such as guanidinium thiocyanate or guanidinium hydrochloride, an alkali perchlorate such as lithium perchlorate, an alkali iodide, magnesium chloride, urea, thiourea, a formamide, or a combination thereof. The concentration of the chaotropic agent can range from about 1 M to about 10 M, such as from about 2.5 M to about 7.5 M, or less than 4.5 M, less than 2 M, or less than 1 M. The chelating agent can be selected from N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy) ethane-N,N,N′,N′-tetraacetic acid (BAPTA), and a phosphonate chelating agent. The concentration of the chelating agent can range from about 10 mM to about 100 mM and/or comprises about 0.5% to about 5% of the lysis reagent. The buffer can be selected from the group consisting of Tris, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, and n-heptyl-β-D-glucopyranoside. The detergent can comprise about 0.1% to about 2% of the lysis reagent, and/or ranges from about 10 mM up to about 100 mM. The lysis reagent can have a pH ranging from about pH 3.0 to about pH 5.5.

In some embodiments, the assays disclosed herein do not utilize a chaotropic agent or a lysis buffer. When a chaotropic agent or lysis buffer is not used, the sample can be contacted with a buffer (or filtering reagent) including, for example, saline (including one or more inorganic salts, such as CaCl₂), MgSO₄, KCl, NaHCO₃, NaCl, etc.), phosphate buffer, Tris buffer, 2-amino-2-hydroxymethyl-1,3-propanediol, HEPES, PBS, citrate buffer, TES, MOPS, PIPES, Cacodylate, SSC, MES, saccharide or disaccharide, or combinations thereof. For example, the buffer can be a commercially available buffer such as Hanks' Balanced Salt Solution available from Sigma Aldrich or TE Buffer available from Fisher BioReagents.

In some embodiments, the alkaline agent can be selected from an alkali metal hydroxide, such as sodium hydroxide or potassium hydroxide. The concentration of the alkaline agent can be about 0.5 N to 5 N.

The binding reagent can promote binding of nucleic acids to the filter, facilitating the removal of non-target material. In some embodiments, the binding reagent can include a binding polymer such as polyacrylic acid (PAA), polyacrylamide (PAM), polyethylene glycol (PEG), poly(sulfobetaine), or a salt, or combinations thereof. In some embodiments, the filtering reagent and/or the washing reagent can include the binding reagent. For example, the binding reagent, the filtering reagent, and/or the washing reagent can include a binding polymer (e.g., PEG 200), buffer, inorganic salt, antioxidant and/or chelating agent, antifoam SE15, sodium azide, disaccharide or disaccharide derivative, carrier protein, detergent, or DMSO. The binding polymer can be present in an amount of at least 10% v/v, at least 20% v/v, at least 30% v/v, and/or less than 60% v/v, less than 40% v/v, less than 30% v/v, less than 20% v/v, or less than 10% v/v or can fall within any range bounded by any of these values, e.g., from 10% to 60% v/v, of the binding reagent, filtering reagent, and/or the washing reagent. The buffer can be selected from the group consisting of Tris, 2-amino-2-hydroxymethyl-1,3-propanediol, HEPES, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The salt, such as NaCl, KCl, or MgCl₂, can be present at a concentration from about 0.05 M to about 1 M, such as from about 0.1 M to about 0.5 M. The antioxidant and/or chelating agent comprises an agent selected from the group consisting of N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy) ethane-N,N,N′,N′-tetraacetic acid (BAPTA), and a phosphonate chelating agent. In some embodiments the antioxidant and/or chelating agent comprises EDTA. In certain embodiments the antioxidant and/or chelating agent comprise 0.2% to about 5%, about 0.2% to about 3%, or about 0.5% to about 2%, or about 0.5% of the binding reagent, filtering reagent, and/or the washing reagent. In some embodiments the concentration of the antioxidant and/or chelating agent in the binding reagent, filtering reagent, or the washing reagent ranges from about 2 mM to about 50 mM or about 5 mM to about 20 mM. In some embodiments, the detergent is an ionic detergent or a non-ionic detergent. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, Brij-35, and n-heptyl-β-D-glucopyranoside. The detergent can comprise about 0.1% to about 2% of the binding reagent, filtering reagent, and/or the washing reagent, and/or ranges from about 10 mM up to about 100 mM. The binding reagent, filtering reagent and/or the washing reagent can have a pH ranging from about pH 6.0 to about pH 8.0 (such as from about 6.5 to about 7.5).

In some embodiments, the eluting reagent can have a pH greater than about 9, greater than about 10, greater than about 11, or greater than about 12. The use of high pH to elute nucleic acid such as DNA is unique especially to the cartridges described herein and provides improved speed and performance of the disclosed methods. Speed is provided by the rapid neutralization of acidic ammonium ions by the high concentration of hydroxide ions. Alkylamines have a pKa ˜10-11 and are immediately deprotonated at pH 12.7, to form the neutral free base on the solid surface, and release the cationic DNA. A further advantage of the high pH is the denaturing effect of KOH on captured DNA or RNA. Acidic functional groups in the heterocyclic bases of DNA or RNA are immediately deprotonated and cannot form Watson-Crick bonds. Double-stranded structures and other secondary structures are disrupted, but can re-nature when neutralized for example, with Tris HCl. This chemical denaturing of captured genomic DNA can be an advantage for isothermal assays that do not undergo the usual heat denaturing of PCR. The cartridges provided herein allow for rapid neutralization of eluted DNA or RNA in KOH followed by reaction with Tris to produce a final pH of about 8.5 for downstream PCR or other nucleic acid assays. In some embodiments, the eluting reagent can have a pH less than about 9, less than about 8.5, or less than about 8. This lower-pH elution of bound DNA or RNA can be an advantage, especially for devices that don't facilitate rapid neutralization of the KOH solution. It is known that RNA is hydrolyzed by high pH, but short exposure times to KOH can provide for good quality RNA. In some examples, the eluting reagent comprises a polyanion, a polycation, ammonia or an alkali metal hydroxide. For example, the eluting reagent may comprise a polyanion such as a carrageenan, a carrier nucleic acid, or a combination thereof.

In some instances, to reduce bubble formation in one or more of the chambers, the detergent Brij may be added to one or more of the reagents described herein.

It is understood that various other reagents and initial volumes can be used for performing an automated PCR panel assay on a sample inserted into the cartridge.

### Exemplary Detection Methods, Results, and Handling of Results

In some embodiments, a computer-based analysis program is used to translate the raw data generated by the detection assay into data of predictive value for a clinician.

Melt Curve analysis can be evaluated by GENEXPERT® Software to determine the presence of PCR product. Melting temperature (Tₘ) and Melt peak height of the curve is calculated automatically by the analysis software. The melt curve is detected as positive if Tₘ falls inside the valid Tₘ range specified for each target nucleic acid. The melt curve is called as negative if melt curve is not in the appropriate Tₘ range. The software automatically calculates the cycle threshold (Ct), Endpoint and Probe check values. Illustrative limit-of-detection concentrations, Ct cut-offs, and methods for determining the same are provided in the examples below.

Before the start of the PCR reaction, the GENEXPERT® System measures the fluorescence signal from the probes to monitor bead rehydration, reaction tube filling, probe integrity, and dye stability. This Probe Check Control (PCC) passes if it meets validated acceptance criteria.

In some embodiments, a computer-based analysis program is used to translate the raw data generated by the detection assay into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

When the GENEXPERT® System is used, the results are interpreted automatically and are shown in a “View Results” window. Positive targets are highlighted in red color, negative targets are highlighted in green color, and indeterminate targets are highlighted in light gray color. Samples with coinfection may appear with positives results for multiple targets. Invalid, Error or No result are highlighted in light gray color.

Exemplary detection methods, results, and handling of results for host biomarker targets are described in US Patent Publication No. 2022/0298572, which is incorporated by reference for this description.

The present disclosure contemplates any method capable of receiving, processing, and transmitting the information to and from laboratories conducting the assays, information provides, medical personal, and subjects. For example, in some embodiments of the present invention, a sample is obtained from a subject and submitted to a testing service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample collected and sent to the testing service, or subjects may collect the sample themselves and directly send it to a testing service. Where the sample includes previously determined biological information, the information may be directly sent to the testing service by the subject (e.g., an information card containing the information may be scanned by a computer and the data transmitted to a computer of the profiling center using an electronic communication systems). Once received by the testing service, the sample is processed and a set of test results is produced, specific for the diagnostic or prognostic information desired for the subject.

The test results can be prepared in a format suitable for interpretation by a treating clinician. For example, rather than providing raw expression data, the prepared format may represent a diagnosis or risk assessment for the subject, with or without recommendations for particular treatment options. The test results may be displayed to the clinician by any suitable method. For example, in some embodiments, the testing service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials, they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, a kit includes primer pairs for amplifying and/or detecting the selected nucleic acid targets, optionally with probes specific for these targets. Such kits can additionally include primers pairs and optional probes for detecting one or more of the above-described host biomarker targets. In some embodiments, these kits can include primers pairs and optional probes for detecting one or more of the above-described controls

In some embodiments, the kit can include any the reagents described above provided with or in one or more GENEXPERT® cartridge(s). See e.g., U.S. Pat. Nos. 5,958,349, 6,403,037, 6,440,725, 6,783,736, 6,818, 185; each of which is herein incorporated by reference for this description). In some embodiments, reagents for measuring detecting target nucleic acids and for detecting host biomarkers are be provided in separate cartridges within a kit.

Any of the kits described here can include, in some embodiments, a receptacle for a sample and/or a swab for collecting a sample.

### EXAMPLES

### Example 1: Three-Phase, Nested Amplification and Multi-Phasic Detection in a Single Cartridge—Method B

This example illustrates an embodiment of above-described Method B: Preamplification (Phase 1)→Nested Amplification 1 (Phase 2)→Detection 1→Nested Amplification 2 (Phase 3)→Detection 2, performed using a GENEXPERT® cartridge and system. This method designed to detect resistance to various tuberculosis drugs, as discussed above.

### Experimental Procedure

Experiments were performed targeting 10 different genes in BCG and 1 internal control (lyophilized beads containing Bacillus globigii spores) making up an 11-plex PCR assay.

Assays were run using 10000, 1000 and 100 CFU/mL BCG in Tris EDTA Triton (TET) buffer to assess the performance of the assay over a wide dynamic range of input template.

Buffer containing BCG cells was added to the GENEXPERT cartridge containing pre-filled PCR reagents (enzyme, primers, probes) and the assay was run in the GENEXPERT® instrument, using pre-defined sample processing and PCR microfluidic steps, which automated the entire PCR and signal detection process.

The assay contained 16 different probes. Two probes to detect BCG have the same fluorescence dye, resulting in a 15-color signal from the PCR, which are detected in two phases as a 10+5 split-signal format.

Real-time and post PCR melt signals were measured twice during the two different phases of nested PCR. The final signal output (both real-time and melt) was obtained as a combination of the signals from both the individual nested PCR phases.

Negative samples (only buffer without BCG) were run regularly to confirm that no carry-over amplicon contamination happened.

More specifically, the following steps were carried out in a cartridge having the layout shown in FIG. 2. Oligonucleotides and enzymes for Preamplification PCR (Phase 1), PCR-1 (Phase 2), and PCR-2 (Phase 3) were provided in three different chambers. The oligos and enzymes can be present in a lyophilized bead form or a liquid PCR cocktail form. When using PCR beads, the liquid PCR cocktail is obtained by dissolving the beads in buffer, using automated microfluidics. GENEXPERT cartridges also contain a separate “master-mix chamber (m-mix chamber), which is directly connected to the reaction tube and is used to (i) store PCR master mixes before loading into the reaction tube or (ii) mixing amplicons from a preceding phase of a PCR with the reaction cocktail of the subsequent PCR, before loading into the reaction tube. Wash and elution buffers were provided in two different chambers. The steps are as follows:
1. Automated sample processing and DNA isolation.
2. The Preamplification PCR master mix (isolated DNA+Preamplification PCR cocktail) from the Preamplification PCR chamber is then pushed into the m-mix chamber for mixing and then aspirated into the reaction chamber for the first time, to perform Preamplification PCR.
3. Preamplification PCR amplicons are then pushed back from the reaction chamber back into the Preamplification PCR chamber (which is securely plugged using a polypropylene frit to prevent any amplicon contamination).
4. Approximately 15% of the Preamplification PCR amplicon is then mixed with the PCR-1 (Nested Amplification 1) reaction cocktail (taken from a PCR-1 chamber) in the m-mix chamber and aspirated into the reaction chamber for a second time (Phase 2), to perform PCR-1 (real-time/melt; first stage of signal detection).
5. PCR-1 amplicons are then pushed back to the PCR-1 chamber (securely plugged with polypropylene frit to prevent any amplicon contamination).
6. The reaction chamber and the m-mix chamber are then washed with TET buffer twice to remove any traces of Preamplification PCR/PCR-1 amplicons and PCR-1 master mix.
7. PCR-2 cocktail (Nested Amplification 2; cocktail stored in a PCR-2 chamber) is now mixed with a second portion of 15% Preamplification PCR amplicon (stored in the Preamplification PCR chamber) in the m-mix chamber, and aspirated into the reaction vessel for a third time, to perform PCR-2 (real-time/melt; second stage of signal detection).

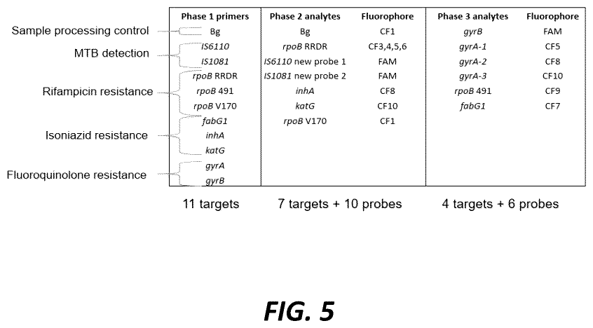

The experimental set-up showing the biomarkers for drug resistance and the phases in which they were detected is shown in FIG. 5. “Phase 1 primers” were the preamplification primers. “Phase 2 analytes” and fluorophores were used for detection in PCR-1 (Nested Amplification/Detection 1). “Phase 3 analytes” and fluorophores were used for detection in PCR-2 (Nested Amplification/Detection 2). This experimental set-up used 16 probes to detect 11 targets.

To determine whether there was carry-over PCR amplicon contamination in the GENEXPERT® modules due to multiple amplicon recycling stages through the cartridge, eleven experiments were conducted over seven days, using 11 different modules. The assays were run with 105 colony-forming units (CFUs) of tuberculosis bacteria, immediately followed by a no-template control run in the same module on the same day over a week to ascertain if the initial run with a high titer CFU, resulted in any amplicon contamination in the GENEXPERT® module, which caused any false positive signals from the no-template controls run in the same modules.

### Results

### Test Sample Results

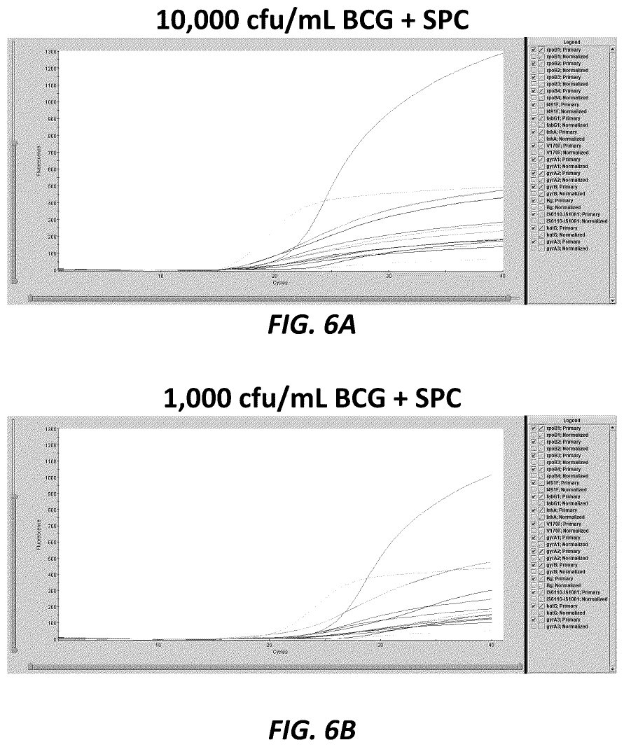

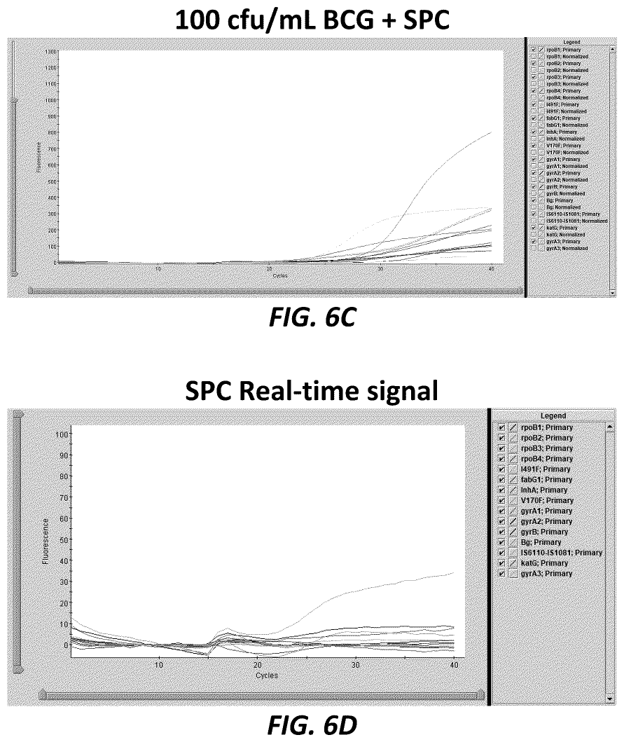

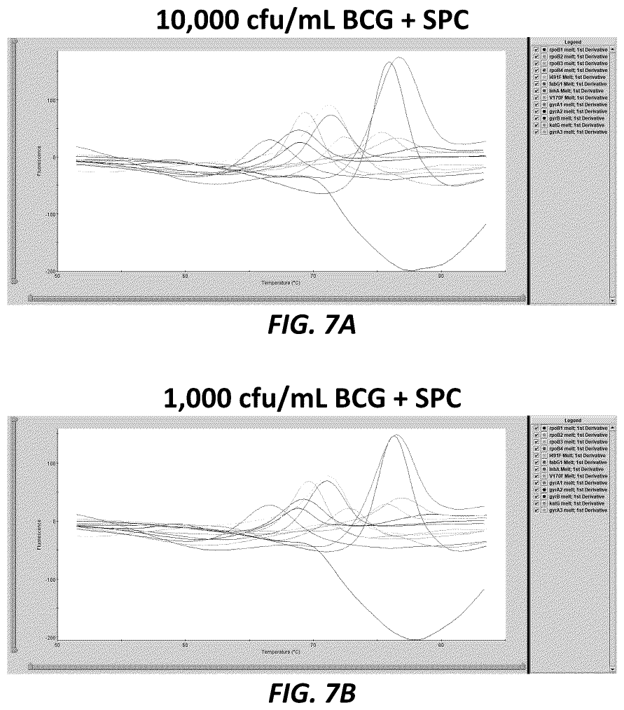

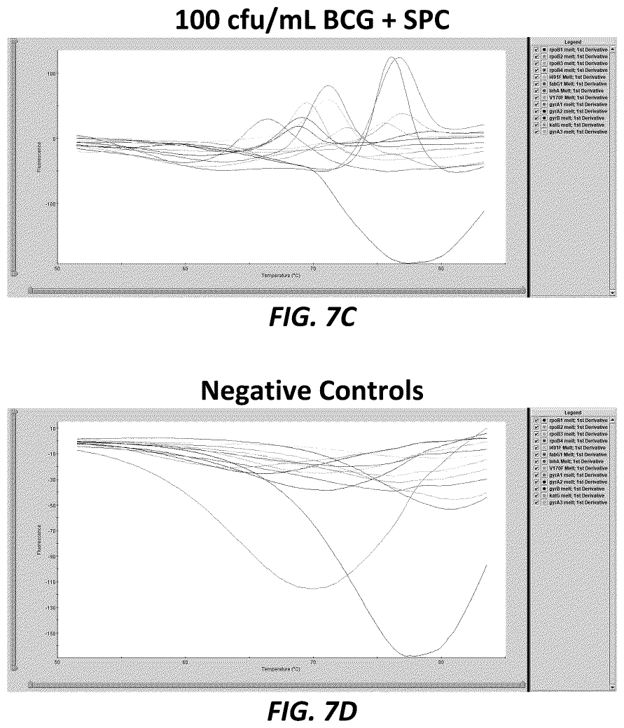

Out of the 15 optical signals, 13 generated both melt and real-time signals and 2 generated only real-time signals as expected, shown in FIGS. 6A-6D and FIGS. 7A-7D. These results demonstrate the effectiveness of Method B.

### Carry-Over Contamination Results

Table 3, below, shows the results of the carry-over contamination study described above. Any amplicon contamination in the GENEXPERT® modules from the initial run, if carried over to the new cartridge with no-template controls, would lead to “false positive” signals from the negative runs.

TABLE 3
No Carry-Over Contamination

| Cartridge | # pos reaction | # neg reaction |
|---|---|---|
| 1 | 5 | 6 |
| 2 | 5 | 6 |
| 3 | 4 | 5 |
| 4 | 2 | 2 |
| 5 | 2 | 2 |
| 6 | 2 | 2 |
| 7 | 3 | 1 |
| 8 | 3 | 1 |
| 9 | 3 | 1 |
| 10 | 2 | 3 |
| 11 | 2 | 3 |
| Total | 33 | 32 |

In all cases, there were no real-time or melt signals observed in the no-template control runs. There was no indication of carry-over contamination in any of the 11 modules and 32 cartridges. Thus, amplicon recycling microfluidics do not increase the risk of any carry-over amplicon contamination in the GENEXPERT® modules.

### Example 2: Three-Phase, Nested Amplification and Multi-Phasic Detection in a Single Cartridge—Method A (Detection 2 by HRM)

This example illustrates an embodiment of above-described Preamplification→Amplification 1→Detection 1→Uniplex Amplification 2→Detection 2 by HRM (high-resolution melt), performed using a GENEXPERT® cartridge and system. This method designed to detect resistance to various tuberculosis drugs, as discussed above.

### Experimental Approach

In this illustrative approach, three gene targets were preamplified together (also termed “Preamplification PCR” or “Phase 1”). Two of the targets were detected using labeled oligonucleotide probes in Nested Asymmetric Amplification 1 (also termed “PCR-1” or “Phase 2”). The third target was detected using EvaGreen HRM in Nested Symmetric Amplification 2 (also termed “PCR-2” or “Phase 3”), as shown in Table 4, below.

TABLE 4
Experimental Set-Up

| | Phase 1 | Phase 2 | Phase 2 | Phase 3 | Phase 3 |
|---|---|---|---|---|---|
| Targets | primers | primers | probes | primers | detection |
| inhA | Outer F | Inner F | gyrA-CF5 | | |
| | and R | and R | | | |
| gyrA | Outer F | Inner F | inhA-CF8 | | |
| | and R | and R | | | |
| rpoB | Quter F | | | Inner F | EvaGreen* |
| | and R | | | and R | |

*Detected in FAM channel

For “Positive” control reaction, 10,000 CFU/mL BCG was employed. For a “Negative” control reaction TET buffer was used.

Three optical channels were used for target detection. Nested Amplification 1 used two channels. Nested Amplification 2 used one channel, which detects signals from the intercalating dye EvaGreen.

### Results

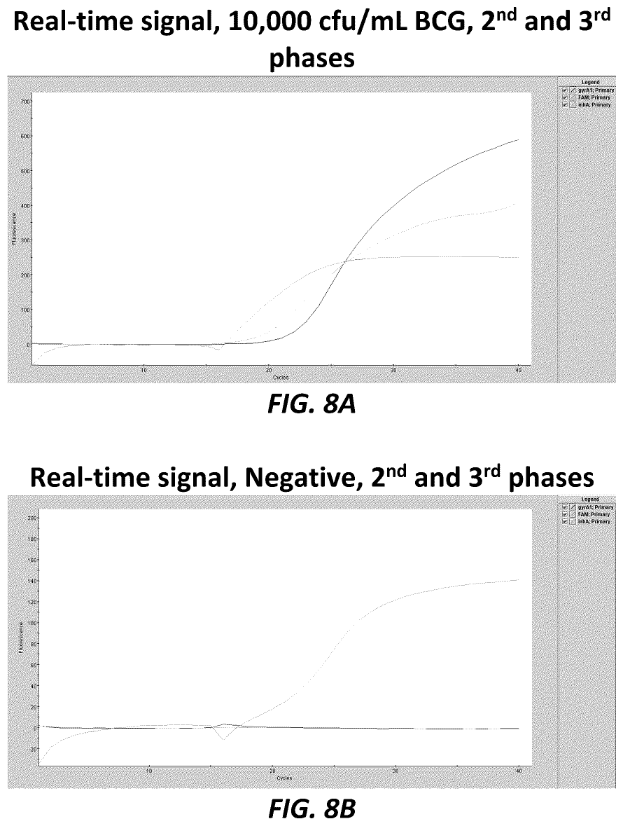

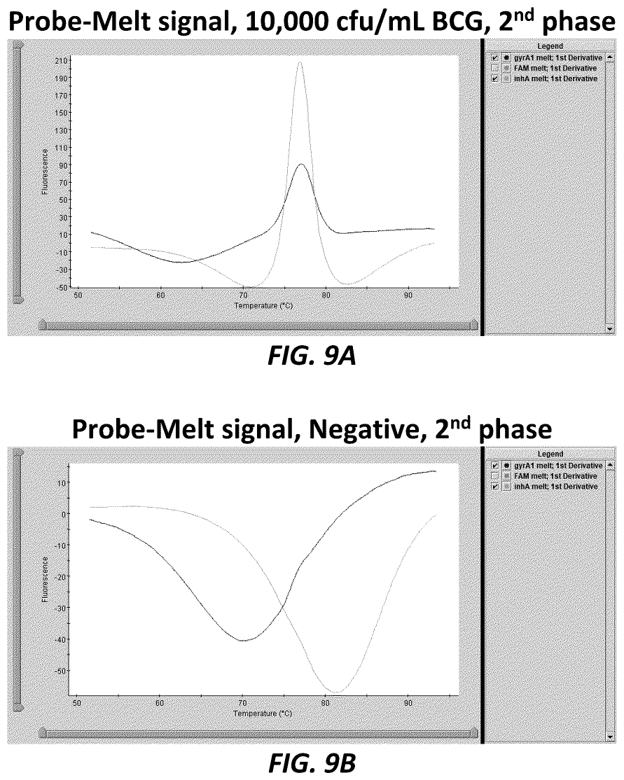

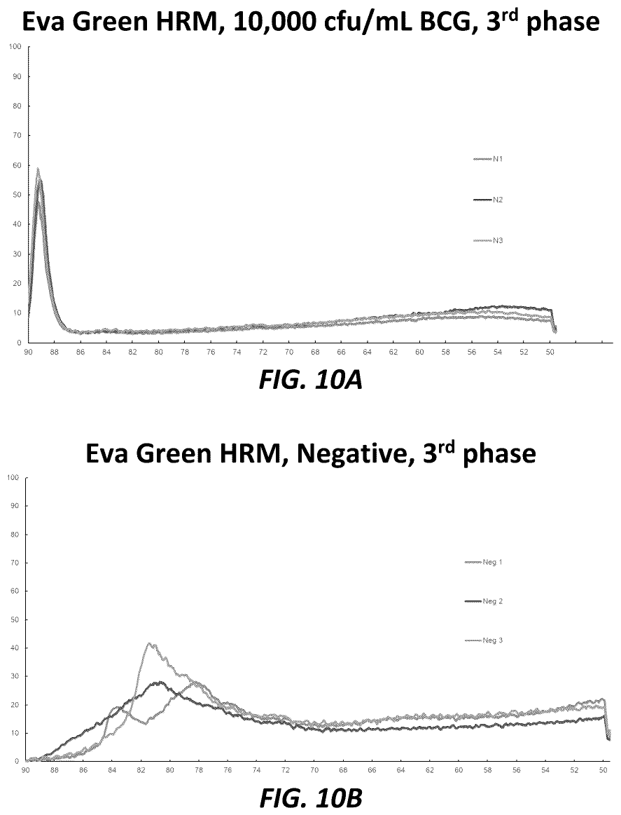

The results are shown in FIGS. 8A-8B, 9A-9B, and 10A-10B. These results demonstrate the effectiveness of Method A.

### Example 3: Detection of 18 Independent Melt and Two Real-Time Signals in a GENEXPERT® Cartridge and System

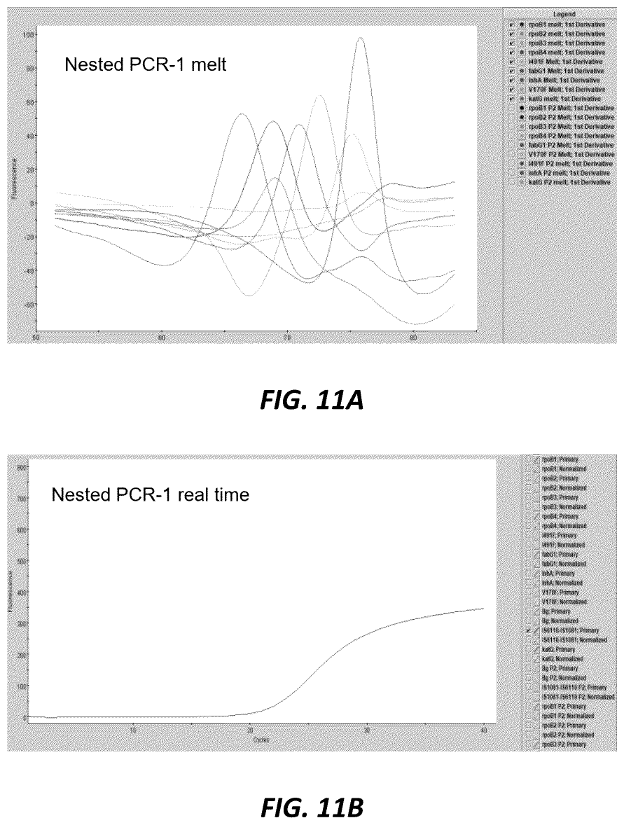

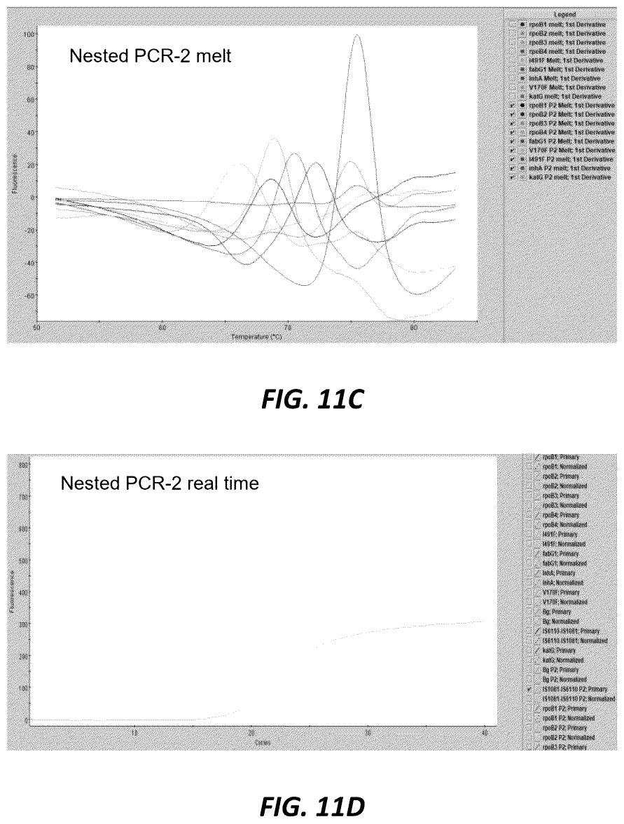

An 8-plex/10-color assay was performed twice over, in the same cartridge to generate 18 independent melt and 2 real-time signals in a 10-color instrument, enabling detection of a total of 20 independent signals in a 10-color instrument. For the melt detection, three different amplicons were amplified from the rpoB gene and one amplicon each was amplified from the fabG1 gene, katG gene, and the inhA promoter region in M. tuberculosis, which were targeted using 9 melt probes. For real-time detection, the IS6110 and IS1081 genes were amplified and detected using two probes in a single channel, since they were conjugated to the same fluorescent dye. The Preamplification PCR contained symmetric primers targeting the different genes, followed by two successive nested PCR phases, containing the same probes and the same asymmetric primers to generate 9 independent melt signal and 1 real-time signal in each successive phase, resulting in a total of 18 melt signals and 2 real-time signals measurable in a single assay. FIGS. 11A-11D show the clear and independent melt and real-time signals that were obtained from the 9 melt probes and two real-time probes (detected in the same optical channel) in the two phases of the assay.

1. A method for detecting target nucleic acids in a sample by nucleic acid amplification, the method comprising:
contacting sample nucleic acids with a set of preamplification primer pairs for amplifying target nucleic acids, wherein the target nucleic acids comprise a first set of target nucleic acids and a second target nucleic acid or set of target nucleic acids;
subjecting the sample nucleic acids and preamplification primer pairs, in solution, to amplification conditions to amplify any target nucleic acids present in the sample nucleic acids to produce a set of double-stranded amplicons;
contacting, in solution, at least a first portion of the double-stranded amplicons with a first set of nested primers and a first set of target-specific probes to form a first reaction mixture;
subjecting the first reaction mixture to amplification conditions in a first amplification to amplify any of the first set of target nucleic acids that are present and conducting a first detection comprising melt analysis or real-time analysis to detect the presence of any of the first set of target nucleic acids that are present;
contacting, in solution, at least a second portion of the double-stranded amplicons with:
a second set of nested, symmetric primers for amplifying the second target nucleic acid and a DNA intercalating dye; or
a second set of nested symmetric or asymmetric primers for amplifying the second set of target nucleic acids and a second set of target-specific real-time or melt probes, respectively;
to form a second reaction mixture;
subjecting the second reaction mixture to amplification conditions in a second amplification to amplify the second target nucleic acid or the second set of target nucleic acids, if present, and conducting a second detection comprising:
high-resolution melt analysis to detect the presence of the second target nucleic acid, if present; or
melt analysis or real-time analysis to detect the presence of any of the second set of target nucleic acids that are present;
wherein the first amplification and first detection and the second amplification and detection are sequential but can be carried out in any order.

2. A cartridge for detecting target nucleic acids in a sample, the cartridge comprising:
a cartridge body comprising a plurality of chambers therein, wherein the plurality of chambers includes:
a sample chamber having at least a fluid outlet in fluid communication with another chamber of the plurality;
an optional lysis chamber in fluidic communication with the sample chamber, optionally wherein the sample chamber and lysis chamber are the same;
a preamplification reagent chamber comprising a first set of preamplification primer pairs for amplifying target nucleic acids, wherein the target nucleic acids comprise a first set of target nucleic acids and a second set of target nucleic acids;
a first reagent chamber comprising a first set of nested primers and a first set of target-specific probes for conducting a first amplification; and
a second reagent chamber comprising a second set of nested primers, wherein:
the second set of nested primers comprises symmetric primers for amplifying a second target nucleic acid, and the second reagent chamber additionally comprises a DNA intercalating dye; or
the second set of nested primers comprises primers for symmetric or asymmetric amplification of a second set of target nucleic acids and a second set of target-specific probes;
a reaction vessel fluidically coupled to the plurality of chambers of the cartridge body and configured for: i) amplification of nucleic acid and ii) detection and identification of one or a plurality of amplification products via melt analysis and/or real-time PCR, wherein the cartridge is configured to carry out multi-phasic detection; and
a filter disposed in a fluidic path between the lysis chamber, if present, or the sample chamber, and the reaction vessel.

3. A cartridge-based method for detecting target nucleic acids in a sample in a cartridge according to claim 2, the method comprising:
placing the sample in the sample chamber of the cartridge;
if the sample comprises cells, lysing cells in the sample with one or more lysis reagents present within at least one of the plurality of chambers or capturing the cells in a filter within the cartridge and lysing the cells by means of ultrasonication to release sample nucleic acids;
if the sample comprises cell-free nucleic acids, capturing the free nucleic acids in a nucleic acid capture chamber and eluting the captured nucleic acid after washing to remove impurities;
contacting sample nucleic acids with preamplification primer pairs within the reaction vessel;
subjecting the sample nucleic acids and preamplification primer pairs, in solution, to amplification conditions to amplify any target nucleic acids present in the sample nucleic acids to produce a set of double-stranded amplicons;
causing the double-stranded amplicons to flow into a first one of the plurality of chambers;
drawing, from the first one of the plurality of chambers, a first portion of the double-stranded amplicons and contacting, in solution, said first portion with a first set of nested primers and a first set of target-specific probes to form a first reaction mixture within the reaction vessel;
subjecting the first reaction mixture to amplification conditions in a first amplification to amplify any of the first set of target nucleic acids that are present and conducting a first detection comprising melt analysis or real-time analysis to detect the presence of any of the first set of target nucleic acids that are present;
causing the first reaction mixture to flow into at least one of the plurality of chambers, and washing the reaction vessel;
drawing, from the first one of the plurality of chambers, a second portion of the double-stranded amplicons and contacting, in solution, said second portion of the double-stranded amplicons with:
a second set of nested, symmetric primers for amplifying the second target nucleic acid and a DNA intercalating dye; or
a second set of nested symmetric or asymmetric primers for amplifying the second set of target nucleic acids and a second set of target-specific probes;
to form a second reaction mixture within the reaction vessel;
subjecting the second reaction mixture to amplification conditions in a second amplification to amplify the second target nucleic acid or the second set of target nucleic acids, if present, and conducting a second detection comprising:
high-resolution melt analysis to detect the presence of the second target nucleic acid, if present; or
melt analysis or real-time analysis to detect the presence of any of the second set of target nucleic acids that are present;
wherein the first amplification and first detection and the second amplification and detection are sequential but can be carried out in any order.

4. The method of claim 1, wherein the second set of nested primers comprises symmetric primers for amplifying the second target nucleic acid and is accompanied by the DNA intercalating dye, and said high-resolution melt analysis is conducted.

5. The method of claim 1, wherein the second set of nested primers comprises asymmetric primers for amplifying the second set of target nucleic acids and is accompanied by the second set of target-specific probes, which are melt detection probes, and melt analysis is conducted.

6. The method of claim 1 wherein the second set of nested primers comprises symmetric primers for amplifying the second set of target nucleic acids and is accompanied by the second set of target-specific probes, which are real-time probes, and real-time analysis is conducted.

7. The method of claim 5, wherein the method detects between 10 and 20 target nucleic acids in each of the first or second reaction mixture.

8. The method, cartridge of claim 7, wherein the method detects between 18 and 20 target nucleic acids in each of the first or second reaction mixture.

9. The method of claim 8, wherein the method detects between 36 and 40 target nucleic acids in a single cartridge.

10. The cartridge of claim 3, wherein the reaction vessel comprises one reaction chamber, and detection of the target nucleic acids is within the one reaction chamber.

11. (canceled)

12. The method of claim 5, wherein the preamplification primers comprise primers specific for one or more of the following drug resistance genes: amikacin resistance genes rrs; aminoglycoside resistance genes rrs and eis; bedaquiline resistance genes atpE and Rv0678; fluoroquinolone resistance genes gyrA and gyrB; capreomycin resistance genes gidB, rrs, and tlyA; clofazimine resistance gene Rv0678; delamanid resistance genes fbiA and ddn; ethionamide resistance genes inhA promoter and ethA; ethambutol resistance gene embB; isoniazid resistance genes fabG1, inhA promoter and katG; linezolid resistance genes rplC and rrl; pyrazinamide resistance gene pncA; rifampin resistance gene rpoB; and streptomycin resistance genes gidB, rrs, and rpsL.

13. The method of claim 5, wherein the preamplification primers comprise primers specific for one or more of the rpoB RRDR gene, rpoB 491 gene, rpoB V170 gene, IS6110 gene, IS1081 gene, fabG1 gene, inhA promoter, katG gene, gyrA gene, gyrB gene, pncA gene, rplC gene, rrl gene, atpE gene and Rv0678 gene of Mycobacterium tuberculosis.

14. The method of claim 13, wherein the preamplification primers comprises primers specific for each of the rpoB RRDR gene, rpoB 491 gene, rpoB V170 gene, IS6110 gene, IS1081 gene, fabG1 gene, inhA promoter, katG gene, gyrA gene, gyrB gene, pncA gene, rplC gene, rrl gene, atpE gene and Rv0678 gene of Mycobacterium tuberculosis.

15. The method of claim 13, wherein the first set of nested primers and the first set of target-specific probes comprise primers and probes specific for one or more of the rpoB RRDR, rpoB 491 and rpoB V170 targets of the rpoB gene, IS6110 gene, IS1081 gene, fabG1 gene, inhA promoter, katG gene, gyrA gene, gyrB gene, Rv0678 gene and pncA gene.

16. The method of claim 14, wherein the first set of nested primers and the first set of target-specific probes comprise primers and probes specific for each of the rpoB RRDR, rpoB 491, and rpoB V170 targets of the rpoB gene, IS6110 gene, IS1081 gene, fabG1 gene, inhA promoter, katG gene, gyrA gene, gyrB gene, Rv0678 gene and pncA gene.

17. The method of claim 13, wherein the second set of nested, symmetric primers is specific for the Rv0678 gene or pncA gene of Mycobacterium tuberculosis.

18. The method of claim 13, wherein the first set of nested primers and the first set of target-specific probes comprise primers and probes specific for one or more of the rpoB RRDR, and rpoB V170 targets of the rpoB gene, IS6110 gene, IS1081 gene, inhA promoter, and katG gene of Mycobacterium tuberculosis.

19. The method of claim 18, wherein the first set of nested primers and the first set of target-specific probes comprise primers and probes specific for each of the rpoB RRDR, and rpoB V170 targets of the rpoB gene, IS6110 gene, IS1081 gene, inhA promoter, and katG gene of Mycobacterium tuberculosis.

20. The method of claim 13, wherein the second set of nested primers and the second set of target-specific probes comprise primers and probes specific for one or more of the rpoB 491 target of the rpoB gene, fabG1 gene, gyrA gene, and gyrB gene of Mycobacterium tuberculosis.

21. The method, cartridge of claim 20, wherein the second set of nested primers and the second set of target-specific probes comprise primers and probes specific for each of the rpoB 491 target of the rpoB gene, fabG1 gene, gyrA gene, and gyrB gene of Mycobacterium tuberculosis.


SEQUENCE LISTING (39 sequences)

SEQ ID NO: 1 - DNA, 21 bp, synthetic construct
  source 1..21: mol_type = other DNA
agcgccgcttcggaccaccag

SEQ ID NO: 2 - DNA, 25 bp, synthetic construct
  source 1..25: mol_type = other DNA
aggcgtcggtgacaaaggccacgta

SEQ ID NO: 3 - DNA, 24 bp, synthetic construct
  source 1..24: mol_type = other DNA
cagcccgacgccgaatcagttgtt

SEQ ID NO: 4 - DNA, 21 bp, synthetic construct
  source 1..21: mol_type = other DNA
ggtgcgggcggtgtcgaggtg

SEQ ID NO: 5 - DNA, 24 bp, synthetic construct
  source 1..24: mol_type = other DNA
cgtggaggcgatcacaccgcagac

SEQ ID NO: 6 - DNA, 24 bp, synthetic construct
  source 1..24: mol_type = other DNA
agctccagcccggcacgctcacgt

SEQ ID NO: 7 - DNA, 22 bp, synthetic construct
  source 1..22: mol_type = other DNA
ccgctggagcagatgggcttgg

SEQ ID NO: 8 - DNA, 23 bp, synthetic construct
  source 1..23: mol_type = other DNA
ccatttcgtcggggtgttcgtcc

SEQ ID NO: 9 - DNA, 24 bp, synthetic construct
  source 1..24: mol_type = other DNA
cacgttacgctcgtggacataccg

SEQ ID NO: 10 - DNA, 26 bp, synthetic construct
  source 1..26: mol_type = other DNA
cggtaaccaggactgaacgggatacg

SEQ ID NO: 11 - DNA, 22 bp, synthetic construct
  source 1..22: mol_type = other DNA
cgccgcttcggaccaccagcac

SEQ ID NO: 12 - DNA, 25 bp, synthetic construct
  source 1..25: mol_type = other DNA
gtgacaaaggccacgtaggcgaacc

SEQ ID NO: 13 - DNA, 21 bp, synthetic construct
  source 1..21: mol_type = other DNA
gccaccgcggggagtttgtcg

SEQ ID NO: 14 - DNA, 22 bp, synthetic construct
  source 1..22: mol_type = other DNA
gtcggttgccgagaccatgggc

SEQ ID NO: 15 - DNA, 21 bp, synthetic construct
  source 1..21: mol_type = other DNA
agcgaccagggctgggccatg

SEQ ID NO: 16 - DNA, 24 bp, synthetic construct
  source 1..24: mol_type = other DNA
atcaacatccggccggtggtcgcc

SEQ ID NO: 17 - DNA, 21 bp, synthetic construct
  source 1..21: mol_type = other DNA
tcacgtgacagaccgccgggc

SEQ ID NO: 18 - DNA, 27 bp, synthetic construct
  source 1..27: mol_type = other DNA
ttacgctcgtggacataccgatttcgg

SEQ ID NO: 19 - DNA, 24 bp, synthetic construct
  source 1..24: mol_type = other DNA
gtttggccccttcagtggctgtgg

SEQ ID NO: 20 - DNA, 25 bp, synthetic construct
  source 1..25: mol_type = other DNA
ggctggaagagctcgtatggcaccg

SEQ ID NO: 21 - DNA, 27 bp, synthetic construct
  source 1..27: mol_type = other DNA
ccatttcgtcggggtgttcgtccatac

SEQ ID NO: 22 - DNA, 21 bp, synthetic construct
  source 1..21: mol_type = other DNA
gcggtcgcgattcgatgttcc

SEQ ID NO: 23 - DNA, 22 bp, synthetic construct
  source 1..22: mol_type = other DNA
ggcgatacttccgctgcgcggc

SEQ ID NO: 24 - DNA, 40 bp, synthetic construct
  source 1..40: mol_type = other DNA
cgaccgcccataaattggctcagctggctggtgacggtcg

SEQ ID NO: 25 - DNA, 44 bp, synthetic construct
  source 1..44: mol_type = other DNA
ggcgcgaaccacgacagcgggttgttctggtccatgaacgcgcc

SEQ ID NO: 26 - DNA, 43 bp, synthetic construct
  source 1..43: mol_type = other DNA
cgcggccgacagttggcgcttgtgggttaaccccgacgccgcg

SEQ ID NO: 27 - DNA, 41 bp, synthetic construct
  source 1..41: mol_type = other DNA
cgcgcgccgggccccagcaccaacagtcggagcttcgcgcg

SEQ ID NO: 28 - DNA, 43 bp, synthetic construct
  source 1..43: mol_type = other DNA
  modified_base 24: mod_base = OTHER; note = uracil
acctgccgcggcgagacgataggttgtaggggtgacggcaggt

SEQ ID NO: 29 - DNA, 38 bp, synthetic construct
  source 1..38: mol_type = other DNA
ccggcgacatcaatggtgctggtgatcgcgtccgccgg

SEQ ID NO: 30 - DNA, 24 bp, synthetic construct
  source 1..24: mol_type = other DNA
cggctgtgggtagcagacctcacc

SEQ ID NO: 31 - DNA, 36 bp, synthetic construct
  source 1..36: mol_type = other DNA
cgcagcggtactcgacgctctgaccgacaagctgcg

SEQ ID NO: 32 - DNA, 39 bp, synthetic construct
  source 1..39: mol_type = other DNA
ccaggggatggcgtagatcgacgcgtcgccgcgccctgg

SEQ ID NO: 33 - DNA, 39 bp, synthetic construct
  source 1..39: mol_type = other DNA
ccaggggatggcgtagatcggcacgtcgccgcgccctgg

SEQ ID NO: 34 - DNA, 39 bp, synthetic construct
  source 1..39: mol_type = other DNA
ccaggggatgccgtagatcgacacgtcgccgcgccctgg

SEQ ID NO: 35 - DNA, 53 bp, synthetic construct
  source 1..53: mol_type = other DNA
  modified_base 28: mod_base = OTHER; note = uracil
  modified_base 13: mod_base = OTHER; note = uracil
ccgagctgatcgtctgaacttcggcgttctttagcacccggttgatagctcgg

SEQ ID NO: 36 - DNA, 25 bp, synthetic construct
  source 1..25: mol_type = other DNA
agcgcttgcggcaaacacggagaaa

SEQ ID NO: 37 - DNA, 26 bp, synthetic construct
  source 1..26: mol_type = other DNA
gcgacaccggcgaatacagagatacc

SEQ ID NO: 38 - DNA, 30 bp, synthetic construct
  source 1..30: mol_type = other DNA
gctccaccgaacaatccgatcttaccgccc

SEQ ID NO: 39 - DNA, 34 bp, synthetic construct
  source 1..34: mol_type = other DNA
  modified_base 29: mod_base = OTHER; note = uracil
cggccgacagacaagctccgtcatttgatcggcc